(19) United States
(12) Patent Application Publication
(10) Pub. No.: US 2011/0283418 A1
(43) Pub. Date: Nov. 17, 2011

(54) Transgenic Plants Having Altered Nitrogen Metabolism

(75) Inventor: Bryan D. McKersie, Raleigh, NC (US)

(73) Assignee: BASF Plant Science Company GmbH, Ludwigshafen (DE)

(21) Appl. No.: 13/145,116

(22) PCT Filed: Jan. 13, 2010
(86) PCT No.: PCT/EP2010/050309

§ 371 Date: Jul. 19, 2011

Related U.S. Application Data
(60) Provisional application No. 61/147,772, filed on Jan. 28, 2009.

(51) Int. Cl. A01H 5/00; A01H 1/06; A01H 5/10

(52) U.S. Cl. 800/288; 800/298; 800/290

(57) ABSTRACT

Polynucleotides are disclosed which are capable of enhancing yield of a plant transformed to contain such polynucleotides. Also provided are methods of using such polynucleotides and transgenic plants and agricultural products, including seeds, containing such polynucleotides as transgenes.

This application claims priority benefit of U.S. provisional patent application Ser. No. 61/147,772, filed Jan. 28, 2009, the entire contents of which are incorporated herein by reference.

### FIELD OF THE INVENTION

This invention relates generally to transgenic plants which overexpress isolated polynucleotides that encode polypeptides active in nitrogen metabolism, in specific plant tissues and organelles, thereby improving yield of said plants.

### BACKGROUND OF THE INVENTION

Population increases and climate change have brought the possibility of global food, feed, and fuel shortages into sharp focus in recent years. Agriculture consumes 70% of water used by people, at a time when rainfall in many parts of the world is declining. In addition, as land use shifts from farms to cities and suburbs, fewer hectares of arable land are available to grow agricultural crops. Agricultural biotechnology has attempted to meet humanity's growing needs through genetic modifications of plants that could increase crop yield, for example, by conferring better tolerance to abiotic stress responses or by increasing biomass.

Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index.

When soil water is depleted or if water is not available during periods of drought, crop yields are restricted. Plant water deficit develops if transpiration from leaves exceeds the supply of water from the roots. The available water supply is related to the amount of water held in the soil and the ability of the plant to reach that water with its root system. Transpiration of water from leaves is linked to the fixation of carbon dioxide by photosynthesis through the stomata. The two processes are positively correlated so that high carbon dioxide influx through photosynthesis is closely linked to water loss by transpiration. As water transpires from the leaf, leaf water potential is reduced and the stomata tend to close in a hydraulic process limiting the amount of photosynthesis. Since crop yield is dependent on the fixation of carbon dioxide in photosynthesis, water uptake and transpiration are contributing factors to crop yield. Plants which are able to use less water to fix the same amount of carbon dioxide or which are able to function normally at a lower water potential have the potential to conduct more photosynthesis and thereby to produce more biomass and economic yield in many agricultural systems.

Agricultural biotechnologists have used assays in model plant systems, greenhouse studies of crop plants, and field trials in their efforts to develop transgenic plants that exhibit increased yield, either through increases in abiotic stress tolerance or through increased biomass. For example, water use efficiency (WUE), is a parameter often correlated with drought tolerance. Studies of a plant's response to desiccation, osmotic shock, and temperature extremes are also employed to determine the plant's tolerance or resistance to abiotic stresses.

An increase in biomass at low water availability may be due to relatively improved efficiency of growth or reduced water consumption. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

Agricultural biotechnologists also use measurements of other parameters that indicate the potential impact of a transgene on crop yield. For forage crops like alfalfa, silage corn, and hay, the plant biomass correlates with the total yield. For grain crops, however, other parameters have been used to estimate yield, such as plant size, as measured by total plant dry weight, above-ground dry weight, above-ground fresh weight, leaf area, stem volume, plant height, rosette diameter, leaf length, root length, root mass, tiller number, and leaf number. Plant size at an early developmental stage will typically correlate with plant size later in development. A larger plant with a greater leaf area can typically absorb more light and carbon dioxide than a smaller plant and therefore will likely gain a greater weight during the same period. There is a strong genetic component to plant size and growth rate, and so for a range of diverse genotypes plant size under one environmental condition is likely to correlate with size under another. In this way a standard environment is used to approximate the diverse and dynamic environments encountered at different locations and times by crops in the field.

Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

Nitrogen plays a crucial role in plant growth. It is the limiting nutrient in the growth of most plant species. The success of a plant's development from the viability of seed through germination and growth are directly related to nitrogen content. Nitrogen is a key component of basic structural elements such as amino acids and resultant proteins, nitrogenous nucleosides, and signaling molecules. Nitrogen metabolism in plants is inextricably linked to carbon metabolism and photosynthesis. Three sources of nitrogen for growth include 1) the assimilation of inorganic N from soil into amino acids; 2) the transfer of amino groups or other organic forms of N from one molecule to another; and 3) the recycling of N from the degradation of proteins, amino acids and other nitrogenous compounds

Urea is widely used throughout the world as a nitrogen fertilizer. However, the nitrogen in urea can only become available to plants through enzymatic hydrolysis by ureases, which convert urea to carbon dioxide and ammonia. Soil microbes contain ureases having two or three subunits designated alpha, beta, and gamma which form a hexamer. Plants also contain hexameric ureases, but the hexamers of plant ureases are made of six identical subunits. Each plant urease subunit contains regions of homology to the microbial urease alpha, beta, and gamma subunits.

Nitrogen in the soil from organic matter and/or commercial fertilizer is taken up by the roots for distribution throughout the plant. Soil-derived nitrogen is usually in the form of nitrate, and must be reduced to ammonia for amino acid and protein synthesis. Ammonia is toxic to plant cells, and must be rapidly converted into amino acids, which are involved in multiple functions in plants. For example, amino acids transport nitrogen between roots, leaves, stems, fruits, etc. In addition, amino acids are precursors in the synthesis of nitrogen-containing compounds such as chlorophyll and enzyme cofactors, like Coenzyme A. furthermore, amino acids are the structural units of protein, and the nitrogen source for secondary compounds such as alkaloids, phenolic acids and cyanogenic compounds.

Amino acid synthetic pathways are branched and interwoven; nitrogen must be distributed to several different amino acid compounds, depending on the needs of the cell at a particular time. Metabolic conditions in the cell may require the synthesis of multiple amino acids, necessitating a highly regulated pathway for the establishment and maintenance of amino acid pools. The enzymes involved in amino acid synthesis are regulated to different degrees. Moreover, amino acids from existing proteins are used to form newly synthesized protein, through the action of proteolytic enzymes. Proteolysis is important in a variety of cellular housekeeping functions, including breakdown abnormal proteins caused by abiotic stresses such as dehydration, mutation, temperature extremes, and free-radical induced damage.

The aromatic amino acids, phenylalanine, tyrosine, and tryptophan not only are required for protein synthesis in plants, but they are also precursors to many aromatic secondary metabolites such as the plant growth regulator auxin, antimicrobial alkaloids, UV-absorbing flavonoids, and polyphenolic compounds like lignin, important for structural support of the xylem. In stressed plant tissue, the increase of aromatic secondary metabolites correlates with increased activity of enzymes (and induced genes) in the aromatic and secondary metabolite pathways, including those of the shikimate pathway. The shikimate pathway can contain 20 percent of a plant's photosynthetically fixed carbon, most of which is shuttled through phenylalanine and tyrosine for secondary metabolites.

Chorismate mutase (EC 5.4.99.5) catalyzes the first step in biosynthesis of aromatic amino acids. Chorismate mutase enzymes have been identified that are monofunctional with or without allosteric control, or bifunctional. When chorismate mutase is bifunctional, the mutase activity may be coupled with prephenate dehydrogenase or 3-deoxyarabinoheptulosonate-7-phosphate synthase. Differential expression of three chorismate mutase genes in Arabidopsis thaliana (CM-1, CM-2, and CM-3) is seen among different tissue types, and in response to environmental stresses. CM-1 and CM-3 are allosterically regulated by free amino acids, while CM-2 is not. Furthermore, CM-1 and CM-3 both have putative plastid targeting sequences, while CM-2 appears to be a cytosolic enzyme, and is the most highly expressed CM RNA in roots.

Nitrogen is also a key component of nucleotides that comprise the hereditary material DNA, RNA, and cofactors such as NADH, (NAD+). Nucleotides can be synthesized de novo from various amino acid classes, or can be recycled through nucleotide salvage pathways, which can save energy. NAD+ (a dinucleotide) can be synthesized de novo from tryptophan or aspartic acid. The main function of NAD+ is to carry electrons through the various redox reactions in cellular metabolism

Although some genes that are involved in stress responses, water use, and/or biomass in plants have been characterized, to date, success at developing transgenic crop plants with improved yield has been limited, and no such plants have been commercialized. There is a need, therefore, to identify additional genes that have the capacity to increase yield of crop plants.

### SUMMARY OF THE INVENTION

The present inventors have discovered that there are three critical components that must be optimized to achieve improvement in plant yield through the modification of nitrogen metabolism: the subcellular targeting of the protein, the level of gene expression and the regulatory properties of the protein. When expressed as described herein, the nitrogen metabolic polynucleotides and polypeptides set forth in Table 1 are capable of improving yield of transgenic plants.

TABLE 1

| | | Nucleotide | |
|---|---|---|---|
| | | SEQ | Amino acid |
| Gene Name | Organism | ID NO: | SEQ ID NO: |
| sll0420 | Synechocystis sp. | 1 | 2 |
| zm07mc17684 | Zea mays | 3 | 4 |
| NP_176922 | A. thaliana | 5 | 6 |
| CAC47052 | Sinorhizobium meliloti | 7 | 8 |
| A7Z9N5 | Bacillus | 9 | 10 |
| | amyloliquefaciens | | |
| Q8UCS8 | Agrobacterium | 11 | 12 |
| | tumefaciens | | |
| B0IYC4 | Rhizobium | 13 | 14 |
| | leguminosarum | | |
| | bv. trifolii | | |
| AAO85883 | Glycine max | 15 | 16 |
| NP_599337 | Corynebacterium | 17 | 18 |
| | glutamicum | | |
| YPR060C | Sacharomyces | 19 | 20 |
| | cereviseae | | |
| GM06MC36749 | G. max | 21 | 22 |
| slr0657 | Synechocystis sp. | 23 | 24 |
| HOM3 | S. cereviseae | 25 | 26 |
| AK1 ARATH | A. thaliana | 27 | 28 |
| A7PA24 | Vitis vinifera | 29 | 30 |
| YMR152W | S. cereviseae | 31 | 32 |
| GM06MC00335 | G. max | 33 | 34 |
| ZM07MC01243 | Z. mays | 35 | 36 |
| b2905 | Escherichia coli | 37 | 38 |
| AT03MContig12206 | A. thaliana | 39 | 40 |
| b2066 | E. coli | 41 | 42 |
| GM06MC13058 | G. max | 43 | 44 |
| OS07MC01669 | Oryza sativa | 45 | 46 |
| ZM07MC11423 | Z. mays | 47 | 48 |
| ZM07MC23131 | Z. mays | 49 | 50 |
| sll0631 | Synechocystis sp. | 51 | 52 |

In one embodiment, the invention provides a transgenic plant transformed with an expression cassette comprising, in operative association, an isolated polynucleotide encoding a promoter and an isolated polynucleotide encoding a full-length polypeptide comprising a urease beta subunit and having urease activity, wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in leaves; an isolated polynucleotide encoding a mitochondrial transit peptide; and an isolated polynucleotide encoding a full-length chorismate mutase polypeptide, wherein the transgenic plant demonstrates increased yield as compared to a wild type plant of the same variety which does not comprise the expression cassette.

In another embodiment, the invention provides a method of increasing yield of a plant by transforming a wild-type plant cell with an expression cassette comprising, in operative association, an isolated polynucleotide encoding a promoter and an isolated polynucleotide encoding a full-length aspartate kinase polypeptide; regenerating transgenic plants from the transformed plant cell; and selecting higher-yielding plants from the regenerated plants.

In another embodiment, the invention provides a method of increasing yield of a plant by transforming a wild-type plant cell with an expression cassette comprising, in operative association, an isolated polynucleotide encoding a promoter; an isolated polynucleotide encoding a mitochondrial transit peptide; and an isolated polynucleotide encoding a full-length protease polypeptide comprising an alcohol dehydrogenase GroES-like domain; regenerating transgenic plants from the transformed plant cell; and selecting higher-yielding plants from the regenerated plants.

In another embodiment, the invention provides a transgenic plant transformed with an expression cassette comprising, in operative association, an isolated polynucleotide encoding a promoter and an isolated polynucleotide encoding a full-length aminomethyltransferase polypeptide comprising a PF01571 domain and a PF08669 domain, wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing expression in leaves, roots or shoots; an isolated polynucleotide encoding a mitochondrial transit peptide; and an isolated polynucleotide encoding a full-length plant uridine/cytidine kinase, wherein the transgenic plant demonstrates increased yield as compared to a wild type plant of the same variety which does not comprise the expression cassette.

In another embodiment, the invention provides a method of increasing yield of a plant by transforming a wild-type plant with an expression cassette comprising, in operative association, an isolated polynucleotide encoding a promoter and an isolated polynucleotide encoding a full-length plant L-aspartate oxidase, regenerating transgenic plants from the transformed plant cell, and selecting higher-yielding plants from the transgenic plants. The expression cassette employed in this embodiment may optionally comprise an isolated polynucleotide encoding a mitochondrial transit peptide.

In a further embodiment, the invention provides a seed produced by the transgenic plants described above, wherein the seed is true breeding for a transgene comprising the expression vectors described above. Plants derived from the seed of the invention demonstrate increased tolerance to an environmental stress, and/or increased plant growth, and/or increased yield, under normal or stress conditions as compared to a wild type variety of the plant.

In a still another aspect, the invention provides products produced by or from the transgenic plants of the invention, their plant parts, or their seeds, such as a foodstuff, feedstuff, food supplement, feed supplement, fiber, cosmetic or pharmaceutical.

The invention further provides certain isolated polynucleotides identified in Table 1, and certain isolated polypeptides identified in Table 1. The invention is also embodied in recombinant vector comprising an isolated polynucleotide of the invention.

In yet another embodiment, the invention concerns a method of producing the aforesaid transgenic plant, wherein the method comprises transforming a plant cell with an expression vector comprising an isolated polynucleotide of the invention, and generating from the plant cell a transgenic plant that expresses the polypeptide encoded by the polynucleotide. Expression of the polypeptide in the plant results in increased tolerance to an environmental stress, and/or growth, and/or yield under normal and/or stress conditions as compared to a wild type variety of the plant.

In still another embodiment, the invention provides a method of increasing a plant's tolerance to an environmental stress, and/or growth, and/or yield. The method comprises the steps of transforming a plant cell with an expression cassette comprising an isolated polynucleotide of Table 1, and generating a transgenic plant from the plant cell, wherein the transgenic plant comprises the polynucleotide.

### BRIEF DESCRIPTION OF THE DRAWINGS

FIG. 1 shows an alignment of the amino acid sequences of urease polypeptides designated sII0420 (SEQ ID NO: 2) and ZM07MC17684 (SEQ ID NO: 4). The alignment was generated using Align X of Vector NTI Advance 10.3.0.

FIG. 2 shows an alignment of the amino acid sequences of chorismate mutase proteins designated YPR060C (SEQ ID NO: 20) and GM06MC36749 (SEQ ID NO: 22). The alignment was generated using Align X of Vector NTI. Advance 10.3.0.

FIG. 3 shows an alignment of the amino acid sequences encoding protease polypeptides designated YMR152W (SEQ ID NO: 32), GM06MC00335 (SEQ ID NO: 34), and ZM07MC01243 (SEQ ID NO: 36). The alignment was generated using Align X of Vector NTI. Advance 10.3.0.

FIG. 4 shows an alignment of the amino acid sequences encoding uridine/cytidine kinase polypeptides designated b2066 (SEQ ID NO: 42), GM06MC13058 (SEQ ID NO: 44), OS07MC01669 (SEQ ID NO: 46), ZM07MC11423 (SEQ ID NO: 48), and ZM07MC23131 (SEQ ID NO: 50). The alignment was generated using Align X of Vector NTI. Advance 10.3.0.

### DETAILED DESCRIPTION OF THE PREFERRED EMBODIMENTS

Throughout this application, various publications are referenced. The disclosures of all of these publications and those references cited within those publications in their entireties are hereby incorporated by reference into this application in order to more fully describe the state of the art to which this invention pertains. The terminology used herein is for the purpose of describing specific embodiments only and is not intended to be limiting. As used herein, “a” or “an” can mean one or more, depending upon the context in which it is used. Thus, for example, reference to “a cell” can mean that at least one cell can be used.

In one embodiment, the invention provides a transgenic plant that overexpresses an isolated polynucleotide identified in Table 1 in the subcellular compartment and tissue indicated herein. The transgenic plant of the invention demonstrates an improved yield as compared to a wild type variety of the plant. As used herein, the term “improved yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bu/acre yield of soybeans or corn derived from a crop comprising plants which are transgenic for the nucleotides and polypeptides of Table 1, as compared with the bu/acre yield from untreated soybeans or corn cultivated under the same conditions, is an improved yield in accordance with the invention.

As defined herein, a “transgenic plant” is a plant that has been altered using recombinant DNA technology to contain an isolated nucleic acid which would otherwise not be present in the plant. As used herein, the term “plant” includes a whole plant, plant cells, and plant parts. Plant parts include, but are not limited to, stems, roots, ovules, stamens, leaves, embryos, meristematic regions, callus tissue, gametophytes, sporophytes, pollen, microspores, and the like. The transgenic plant of the invention may be male sterile or male fertile, and may further include transgenes other than those that comprise the isolated polynucleotides described herein.

As used herein, the term “variety” refers to a group of plants within a species that share constant characteristics that separate them from the typical form and from other possible varieties within that species. While possessing at least one distinctive trait, a variety is also characterized by some variation between individuals within the variety, based primarily on the Mendelian segregation of traits among the progeny of succeeding generations. A variety is considered “true breeding” for a particular trait if it is genetically homozygous for that trait to the extent that, when the true-breeding variety is self-pollinated, a significant amount of independent segregation of the trait among the progeny is not observed. In the present invention, the trait arises from the transgenic expression of one or more isolated polynucleotides introduced into a plant variety. As also used herein, the term “wild type variety” refers to a group of plants that are analyzed for comparative purposes as a control plant, wherein the wild type variety plant is identical to the transgenic plant (plant transformed with an isolated polynucleotide in accordance with the invention) with the exception that the wild type variety plant has not been transformed with an isolated polynucleotide of the invention. The term “wild type” as used herein refers to a plant cell, seed, plant component, plant tissue, plant organ, or whole plant that has not been genetically modified with an isolated polynucleotide in accordance with the invention.

The term “control plant” as used herein refers to a plant cell, an explant, seed, plant component, plant tissue, plant organ, or whole plant used to compare against transgenic or genetically modified plant for the purpose of identifying an enhanced phenotype or a desirable trait in the transgenic or genetically modified plant. A “control plant” may in some cases be a transgenic plant line that comprises an empty vector or marker gene, but does not contain the recombinant polynucleotide of interest that is present in the transgenic or genetically modified plant being evaluated. A control plant may be a plant of the same line or variety as the transgenic or genetically modified plant being tested, or it may be another line or variety, such as a plant known to have a specific phenotype, characteristic, or known genotype. A suitable control plant would include a genetically unaltered or non-transgenic plant of the parental line used to generate a transgenic plant herein.

As defined herein, the term “nucleic acid” and “polynucleotide” are interchangeable and refer to RNA or DNA that is linear or branched, single or double stranded, or a hybrid thereof. The term also encompasses RNA/DNA hybrids. An “isolated” nucleic acid molecule is one that is substantially separated from other nucleic acid molecules which are present in the natural source of the nucleic acid (i.e., sequences encoding other polypeptides). For example, a cloned nucleic acid is considered isolated. A nucleic acid is also considered isolated if it has been altered by human intervention, or placed in a locus or location that is not its natural site, or if it is introduced into a cell by transformation. Moreover, an isolated nucleic acid molecule, such as a cDNA molecule, can be free from some of the other cellular material with which it is naturally associated, or culture medium when produced by recombinant techniques, or chemical precursors or other chemicals when chemically synthesized. While it may optionally encompass untranslated sequence located at both the 3′ and 5′ ends of the coding region of a gene, it may be preferable to remove the sequences which naturally flank the coding region in its naturally occurring replicon.

As used herein, the term “environmental stress” refers to a sub-optimal condition associated with salinity, drought, nitrogen, temperature, metal, chemical, pathogenic, or oxidative stresses, or any combination thereof. As used herein, the term “drought” refers to an environmental condition where the amount of water available to support plant growth or development is less than optimal. As used herein, the term “fresh weight” refers to everything in the plant including water. As used herein, the term “dry weight” refers to everything in the plant other than water, and includes, for example, carbohydrates, proteins, oils, and mineral nutrients.

Any plant species may be transformed to create a transgenic plant in accordance with the invention. The transgenic plant of the invention may be a dicotyledonous plant or a monocotyledonous plant. For example and without limitation, transgenic plants of the invention may be derived from any of the following diclotyledonous plant families: Leguminosae, including plants such as pea, alfalfa and soybean; Umbelliferae, including plants such as carrot and celery; Solanaceae, including the plants such as tomato, potato, aubergine, tobacco, and pepper; Cruciferae, particularly the genus Brassica, which includes plant such as oilseed rape, beet, cabbage, cauliflower and broccoli); and A. thaliana; Compositae, which includes plants such as lettuce; Malvaceae, which includes cotton; Fabaceae, which includes plants such as peanut, and the like. Transgenic plants of the invention may be derived from monocotyledonous plants, such as, for example, wheat, barley, sorghum, millet, rye, triticale, maize, rice, oats and sugarcane. Transgenic plants of the invention are also embodied as trees such as apple, pear, quince, plum, cherry, peach, nectarine, apricot, papaya, mango, and other woody species including coniferous and deciduous trees such as poplar, pine, sequoia, cedar, oak, and the like. Especially preferred are A. thaliana, Nicotiana tabacum, rice, oilseed rape, canola, soybean, corn (maize), cotton, and wheat.

In one embodiment, the invention provides a transgenic plant with an expression cassette comprising, in operative association, an isolated polynucleotide encoding a promoter and an isolated polynucleotide encoding a full-length urease beta subunit polypeptide. In a second step, transgenic plantlets are regenerated from the transformed plant cell. In a third step, the transgenic plantlets are subjected to a yield-related assay, and higher-yielding plants are selected from the regenerated transgenic plants.

As indicated in Table 2 below, when the Synechocystis urease gene sII0420 (SEQ ID NO: 1) is expressed under control of the pcUbi promoter, plants demonstrate improved yield under water-limiting conditions. The sII0420 urease polypeptide (SEQ ID NO:2) comprises a urease beta subunit domain (PF00699) from amino acid 5 to amino acid 104 of SEQ ID NO:2. The Z. mays urease set forth in SEQ ID NO:4 comprises a urease beta domain from amino acid 132 to amino acid 231, a urease alpha domain (PF00449) from amino acid 268 to amino acid 385, and a urease gamma domain (PF00547) from amino acid 1 to amino acid 100 of SEQ ID NO:4, respectively. The A. thaliana urease of SEQ ID NO:6 comprises a urease beta domain from amino acid 133 to amino acid 232, a urease alpha domain from amino acid 270 to amino acid 390, and a urease gamma domain from amino acid 1 to amino acid 100 of SEQ ID NO:6, respectively. The S. meliloti urease of SEQ ID NO:8 comprises a urease beta domain from amino acid 2 to amino acid 101. The B. amyloliquefaciens urease of SEQ ID NO:10 comprises a urease beta domain from amino acid 2 to amino acid 101. The A. tumifaciens urease of SEQ ID NO:12 comprises a urease beta domain from amino acid 2 to amino acid 101. The R. leguminosarum urease of SEQ ID NO:14 comprises a urease beta domain from amino acid 2 to amino acid 101. The G. max urease of SEQ ID NO:16 comprises a urease beta domain from amino acid 133 to amino acid 232, a urease alpha domain from amino acid 270 to amino acid 390, and a urease gamma domain from amino acid 1 to amino acid 100 of SEQ ID NO:16, respectively. The C. glutamicum urease of SEQ ID NO:18 comprises a urease beta domain from amino acid 2 to amino acid 101.

In accordance with the invention, any urease polypeptide comprising a urease beta subunit domain may be employed in the method of this embodiment. Preferably, the method of this embodiment employs a polynucleotide encoding a full-length urease polypeptide comprising a beta subunit domain. More preferably, the polynucleotide employed in this embodiment encodes a urease polypeptide comprises a beta subunit domain selected from the group consisting of amino acid 5 to amino acid 104 of SEQ ID NO:2; amino acid 132 to amino acid 231 of SEQ ID NO:4; amino acid 133 to amino acid 232 of SEQ ID NO:6; amino acid 2 to amino acid 101 of SEQ ID NO:8; amino acid 2 to amino acid 101 of SEQ ID NO:10; amino acid 2 to amino acid 101 of SEQ ID NO:12; amino acid 2 to amino acid 101 of SEQ ID NO:14; amino acid 133 to amino acid 232 of SEQ ID NO:16; and amino acid 2 to amino acid 101 of SEQ ID NO:18. Most preferably, the polynucleotide employed in the method of this embodiment encodes urease having a sequence comprising amino acids 1 to 105 of SEQ ID NO: 2; amino acid 1 to amino acid 385 of SEQ ID NO:4; amino acid 1 to amino acid 838 of SEQ ID NO:6; amino acid 1 to amino acid 101 of SEQ ID NO:8; amino acid 1 to amino acid 124 of SEQ ID NO:10; amino acid 1 to amino acid 101 of SEQ ID NO:12; amino acid 1 to amino acid 101 of SEQ ID NO:14; amino acid 1 to amino acid 837 of SEQ ID NO:16; or amino acid 1 to amino acid 162 of SEQ ID NO:18.

In another embodiment, the invention provides a transgenic plant transformed with an expression cassette comprising, in operative association, an isolated polynucleotide encoding a promoter; an isolated polynucleotide encoding a mitochondrial transit peptide; and an isolated polynucleotide encoding a full-length chorismate mutase polypeptide, wherein the transgenic plant demonstrates increased yield as compared to a wild type plant of the same variety which does not comprise the expression cassette.

As indicated in Table 3 below, when the S. cerevisiae chorismate mutase gene YPR060C (SEQ ID NO: 19) is targeted to the mitochondria under control of the USP promoter, plants demonstrate improved response to water-limiting conditions. The YPR060C chorismate mutase polypeptide comprises a CM_{—}2 signature sequence from amino acid 16 to amino acid 85 of SEQ ID NO:20. The G. max chorismate mutase polypeptide of SEQ ID NO:22 comprises a CM_{—}2 signature sequence from amino acid 21 to amino acid 88 of SEQ ID NO:22.

In accordance with the invention, the expression cassette of this embodiment may comprise any polynucleotide encoding a chorismate mutase polypeptide Preferably, the expression cassette of this embodiment comprises a polynucleotide encoding a chorismate mutase polypeptide comprising a CM_{—}2 domain selected from the group consisting of amino acids 16 to 85 of SEQ ID NO: 20 and amino acids 21 to 88 of SEQ ID NO: 22. More preferably, the method of this embodiment employs a polynucleotide encoding a chorismate mutase polypeptide comprising amino acids 1 to 256 of SEQ ID NO: 20 or amino acids 1 to 261 of SEQ ID NO: 22.

In another embodiment, the invention provides a method of increasing yield of a plant by transforming a wild-type plant cell with an expression cassette comprising, in operative association, an isolated polynucleotide encoding a promoter and an isolated polynucleotide encoding a full-length aspartate kinase polypeptide; regenerating transgenic plants from the transformed plant cell; and selecting higher-yielding plants from the regenerated plants.

As shown in Table 4 below, the Synechocystis aspartate kinase gene slr0657 (SEQ ID NO: 23) under the control of the pcUbi promoter, transgenic plants demonstrate greater biomass compared to the control plants. The amino acid kinase family motif (PF00696) is present in slr0657 at residues 2-232 of SEQ ID NO: 24. Additionally, the ACT domain (PF01842) is present in slr0657 from amino acid residues 272-338, 359-417, 447-517 and 531-589 of SEQ ID NO: 24.

Preferably, the method of this embodiment employs a polynucleotide encoding a polypeptide having aspartate kinase activity and comprising a PF00696 domain. More preferably, the method of this embodiment employs a polynucleotide encoding an aspartate kinase polypeptide comprising amino acids 2 to 232 of SEQ ID NO: 24 and at least one PF01842 domain selected from the group consisting of amino acids 195-264 of SEQ ID NO:24, amino acids 359-417 of SEQ ID NO: 24, amino acids 447-517 of SEQ ID NO: 24 and amino acids 531-589 of SEQ ID NO: 24. Most preferably, the method of this embodiment employs a polynucleotide encoding an aspartate kinase protein having a sequence comprising amino acids 1 to 600 f SEQ ID NO: 24; amino acids 1 to 527 of SEQ ID NO:26, amino acids 1 to 569 of SEQ ID NO:28, or amino acids 1 to 569 of SEQ ID NO:30.

In another embodiment, the invention provides a method of increasing yield of a plant by transforming a wild-type plant cell with an expression cassette comprising, in operative association, an isolated polynucleotide encoding a promoter; an isolated polynucleotide encoding a mitochondrial transit peptide; and an isolated polynucleotide encoding a full-length protease polypeptide comprising an alcohol dehydrogenase GroES-like domain; regenerating transgenic plants from the transformed plant cell; and selecting higher-yielding plants from the regenerated plants.

As set forth in Table 5 below, when the S. cerevisiae gene YMR152W (SEQ ID NO: 31) is targeted to the mitochondria under the control of the USP promoter, plants demonstrate improved response to cycling drought conditions. Gene YMR152W encodes a protease which comprises the alcohol dehydrogenase GroES-like (ADH_N) domain designated PF08240, at amino acids 35 to 123 of SEQ ID NO: 32. The G. max protease set forth in SEQ ID NO: 34 comprise an ADH_N domain at amino acids 111 to 202. The Z. mays protease set forth in SEQ ID NO: 36 comprise an ADH_N domain at amino acids 28 to 119.

In accordance with the invention, the method of this embodiment may employ any polynucleotide encoding a protease polypeptide that comprises an ADH-N domain. Preferably, the polynucleotide employed in the method encodes a protease polypeptide comprising an ADH_N domain is selected from the group consisting of amino acids 35 to 123 of SEQ ID NO: 32, amino acids 111 to 202 of SEQ ID NO: 34; and amino acids 28 to 119 of SEQ ID NO: 36. Most preferably, the polynucleotide employed in the method of this embodiment encodes a protease having a sequence comprising amino acids 1 to 365 f SEQ ID NO: 32, amino acids 1 to 392 of SEQ ID NO: 34, or amino acids 1 to 251 of SEQ ID NO:36.

In another embodiment, the invention provides a transgenic plant transformed with an expression cassette comprising, in operative association, an isolated polynucleotide encoding a promoter and an isolated polynucleotide encoding a full-length aminomethyltransferase polypeptide comprising a PF01571 domain and a PF08669 domain, wherein the transgenic plant demonstrates increased yield as compared to a wild type plant of the same variety which does not comprise the expression cassette.

As set forth in Table 6 below, when the E. coli gene b2905 (SEQ ID NO: 37) is expressed in A. thaliana with no subcellular targeting signal under the control of the Super promoter, plants demonstrate improved response to cycling drought and well watered conditions. Gene b2905 encodes a aminomethyltransferase which comprises two domains: the aminomethyltranferase folate-binding domain designated PF01571, at amino acids 46 to 254 of SEQ ID NO:38; and the glycine cleavage T-protein C-terminal barrel domains designated PF08669 at amino acids 262 to 354 of SEQ ID NO:38.

The transgenic plant of this embodiment may comprise any polynucleotide encoding an aminomethyltransferase polypeptide. Preferably, the aminomethyltransferase polypeptide expressed in the transgenic plant of this embodiment comprises a PF01571 domain selected from the group consisting of amino acids 46 to 254 of SEQ ID NO:38; amino acids 81 to 296 of SEQ ID NO:40; and a PF08669 domain. selected from the group consisting of amino acids 305 to 397 of SEQ ID NO: 40; More preferably, the transgenic plant of this embodiment comprises a polynucleotide encoding an aminomethyltransferase having a sequence comprising amino acids 1 to 364 of SEQ ID NO: 38; amino acids 1 to 408 of SEQ ID NO: 40.

In another embodiment, the invention provides a transgenic plant transformed with an expression cassette comprising, in operative association, an isolated polynucleotide encoding a promoter capable of enhancing expression in leaves, roots or shoots; an isolated polynucleotide encoding a mitochondrial transit peptide; and an isolated polynucleotide encoding a full-length plant uridine/cytidine kinase, wherein the transgenic plant demonstrates increased yield as compared to a wild type plant of the same variety which does not comprise the expression cassette.

As set forth in Table 7 below, when the E. coli gene b2066 (SEQ ID NO: 41) is expressed with mitochondrial targeting signal under the control of the USP promoter or the Super promoter, plants demonstrate improved response under water limiting conditions. Gene b2066 encodes a uridine kinase comprises the Phosphoribulokinase/Uridine kinase family domain designated PF00485, at amino acids 28 to 218 of SEQ ID NO:42.

The transgenic plant of this embodiment may comprise any polynucleotide encoding a plant uridine kinase polypeptide. Preferably, the transgenic plant of this embodiment comprises a polynucleotide encoding a uridine kinase polypeptide comprising the domain designated PF00485 selected from the group consisting of amino acids 55 to 242 of SEQ ID NO: 42; amino acids 1 to 121 of SEQ ID NO: 46, amino acids 67 to 241 of SEQ ID NO: 48, amino acids 52 to 228 of SEQ ID NO: 50. More preferably, the transgenic plant of this embodiment comprises a polynucleotide encoding a plant uridine kinase having a sequence comprising amino acids 1 to 231 of SEQ ID NO:42, amino acids 1 to 476 of SEQ ID NO: 44, amino acids 1 to 496 of SEQ ID NO: 46, amino acids 1 to 251 of SEQ ID NO:48, or amino acids 1 to 228 of SEQ ID NO:50.

In another embodiment, the invention provides a method of increasing yield of a plant by transforming a wild-type plant with an expression cassette comprising, in operative association, an isolated polynucleotide encoding a promoter and an isolated polynucleotide encoding a full-length plant L-aspartate oxidase, regenerating transgenic plants from the transformed plant cell, and selecting higher-yielding plants from the transgenic plants. The expression cassette employed in this embodiment may optionally comprise an isolated polynucleotide encoding a mitochondrial transit peptide.

As set forth in Table 8 below, when the Synechocystis gene sII0631 (SEQ ID NO: 52) controlled by the PCUbi promoter were significantly larger than control plants that did not express the sII0631 gene under water-limiting conditions. This effect was seen in transgenic plants with no sub cellular targeting under the stress of water-limiting conditions. Gene sII0631 encodes L-aspartate oxidase characterized by having a FAD binding domain designated PF00890 found at amino acids 7 to 381 of SEQ ID NO:52.

The method of this embodiment may employ an expression cassette comprising any polynucleotide encoding an L-aspartate oxidase polypeptide. Preferably, the polynucleotide employed in the method of this embodiment encodes an L-aspartate oxidase polypeptide comprising a PF00890 domain. More preferably, the polynucleotide employed in the method of this embodiment encodes an L-aspartate oxidase polypeptide comprising amino acids 7 to 381 of SEQ ID NO: 52. Most preferably, the method of this embodiment employs a polynucleotide encoding L-aspartate oxidase having a sequence comprising amino acids 1 to 553 of SEQ ID NO:52.

The invention further provides a seed which is true breeding for the expression cassettes (also referred to herein as “transgenes”) described herein, wherein transgenic plants grown from said seed demonstrate increased yield as compared to a wild type variety of the plant. The invention also provides a product produced by or from the transgenic plants expressing the polynucleotide, their plant parts, or their seeds. The product can be obtained using various methods well known in the art. As used herein, the word “product” includes, but not limited to, a foodstuff, feedstuff, a food supplement, feed supplement, fiber, cosmetic or pharmaceutical. Foodstuffs are regarded as compositions used for nutrition or for supplementing nutrition. Animal feedstuffs and animal feed supplements, in particular, are regarded as foodstuffs. The invention further provides an agricultural product produced by any of the transgenic plants, plant parts, and plant seeds. Agricultural products include, but are not limited to, plant extracts, proteins, amino acids, carbohydrates, fats, oils, polymers, vitamins, and the like.

The invention also provides an isolated polynucleotide which has a sequence selected from the group consisting of SEQ ID NO: 3; SEQ ID NO: 5; SEQ ID NO: 21; SEQ ID NO:33, SEQ ID NO:35, SEQ ID NO: 39; SEQ ID NO: 43; SEQ ID NO: 45, SEQ ID NO: 47 and SEQ ID NO: 49. Also encompassed by the invention is an isolated polynucleotide encoding a polypeptide having an amino acid sequence selected from the group consisting of SEQ ID NO: 4; SEQ ID NO: 6; SEQ ID NO: 22; SEQ ID NO:34, SEQ ID NO:36, SEQ ID NO: 40; SEQ ID NO: 44; SEQ ID NO: 46, SEQ ID NO: 48, and SEQ ID NO: 50. A polynucleotide of the invention can be isolated using standard molecular biology techniques and the sequence information provided herein, for example, using an automated DNA synthesizer.

The isolated polynucleotides of the invention include homologs of the polynucleotides of Table 1. “Homologs” are defined herein as two nucleic acids or polypeptides that have similar, or substantially identical, nucleotide or amino acid sequences, respectively. Homologs include allelic variants, analogs, and orthologs, as defined below. As used herein, the term “analogs” refers to two nucleic acids that have the same or similar function, but that have evolved separately in unrelated organisms. As used herein, the term “orthologs” refers to two nucleic acids from different species, but that have evolved from a common ancestral gene by speciation. The term homolog further encompasses nucleic acid molecules that differ from one of the nucleotide sequences shown in Table 1 due to degeneracy of the genetic code and thus encode the same polypeptide.

To determine the percent sequence identity of two amino acid sequences (e.g., one of the polypeptide sequences of Table 1 and a homolog thereof), the sequences are aligned for optimal comparison purposes (e.g., gaps can be introduced in the sequence of one polypeptide for optimal alignment with the other polypeptide or nucleic acid). The amino acid residues at corresponding amino acid positions are then compared. When a position in one sequence is occupied by the same amino acid residue as the corresponding position in the other sequence then the molecules are identical at that position. The same type of comparison can be made between two nucleic acid sequences.

Preferably, the isolated amino acid homologs, analogs, and orthologs of the polypeptides of the present invention are at least about 50-60%, preferably at least about 60-70%, and more preferably at least about 70-75%, 75-80%, 80-85%, 85-90%, or 90-95%, and most preferably at least about 96%, 97%, 98%, 99%, or more identical to an entire amino acid sequence identified in Table 1. In another preferred embodiment, an isolated nucleic acid homolog of the invention comprises a nucleotide sequence which is at least about 40-60%, preferably at least about 60-70%, more preferably at least about 70-75%, 75-80%, 80-85%, 85-90%, or 90-95%, and even more preferably at least about 95%, 96%, 97%, 98%, 99%, or more identical to a nucleotide sequence shown in Table 1.

For the purposes of the invention, the percent sequence identity between two nucleic acid or polypeptide sequences is determined using Align 2.0 (Myers and Miller, CABIOS (1989) 4:11-17) with all parameters set to the default settings or the Vector NTI Advance 10.3.0 (PC) software package (Invitrogen, 1600 Faraday Ave., Carlsbad, Calif. 92008). For percent identity calculated with Vector NTI, a gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide.

Nucleic acid molecules corresponding to homologs, analogs, and orthologs of the polypeptides listed in Table 1 can be isolated based on their identity to said polypeptides, using the polynucleotides encoding the respective polypeptides or primers based thereon, as hybridization probes according to standard hybridization techniques under stringent hybridization conditions. As used herein with regard to hybridization for DNA to a DNA blot, the term “stringent conditions” refers to hybridization overnight at 60° C. in 10× Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10×Denhart's solution, 6×SSC, 0.5% SDS and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for performing nucleic acid hybridizations are well known in the art.

The isolated polynucleotides employed in the invention may be optimized, that is, genetically engineered to increase its expression in a given plant or animal. To provide plant optimized nucleic acids, the DNA sequence of the gene can be modified to: 1) comprise codons preferred by highly expressed plant genes; 2) comprise an A+T content in nucleotide base composition to that substantially found in plants; 3) form a plant initiation sequence; 4) to eliminate sequences that cause destabilization, inappropriate polyadenylation, degradation and termination of RNA, or that form secondary structure hairpins or RNA splice sites; or 5) elimination of antisense open reading frames. Increased expression of nucleic acids in plants can be achieved by utilizing the distribution frequency of codon usage in plants in general or in a particular plant. Methods for optimizing nucleic acid expression in plants can be found in EPA 0359472; EPA 0385962; PCT Application No. WO 91/16432; U.S. Pat. No. 5,380,831; U.S. Pat. No. 5,436,391; Perlack et al., 1991, Proc. Natl. Acad. Sci. USA 88:3324-3328; and Murray et al., 1989, Nucleic Acids Res. 17:477-498.

The invention further provides a recombinant expression vector which comprises an expression cassette selected from the group consisting of a) an expression cassette comprising, in operative association, an isolated polynucleotide encoding a promoter, an isolated polynucleotide encoding a subcellular targeting peptide, and an isolated polynucleotide encoding a full-length phosphatidate cytidylyltransferase polypeptide; b) an expression cassette comprising, in operative association, an isolated polynucleotide encoding a promoter capable of enhancing expression in leaves, an isolated polynucleotide encoding a mitochondrial transit peptide, and an isolated polynucleotide encoding an acyl-carrier protein; and c) an expression cassette comprising, in operative association, an isolated polynucleotide encoding a promoter, an isolated polynucleotide encoding a subcellular targeting peptide; and an isolated polynucleotide encoding an acyltransferase polypeptide.

In another embodiment, the recombinant expression vector of the invention comprises an isolated polynucleotide having a sequence selected from the group consisting of SEQ ID NO: 3; SEQ ID NO: 5; SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23; SEQ ID NO:25, SEQ ID NO: 27; SEQ ID NO: 29; SEQ ID NO: 31, SEQ ID NO: 33; SEQ ID NO: 35, SEQ ID NO: 37, SEQ ID NO: 39, SEQ ID NO: 41, SEQ ID NO:43, SEQ ID NO: 45; SEQ ID NO: 47; SEQ ID NO: 49, SEQ ID NO: 51 In addition, the recombinant expression vector of the invention comprises an isolated polynucleotide encoding a polypeptide having an amino acid sequence selected from the group consisting SEQ ID NO: 4; SEQ ID NO: 6; SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24; SEQ ID NO:26, SEQ ID NO: 28; SEQ ID NO: 30; SEQ ID NO: 32, SEQ ID NO: 34; SEQ ID NO: 36, SEQ ID NO: 38, SEQ ID NO: 40, SEQ ID NO: 42, SEQ ID NO:44, SEQ ID NO: 46; SEQ ID NO: 48; SEQ ID NO: 50 and SEQ ID NO: 52.

The recombinant expression vector of the invention may also include one or more regulatory sequences, selected on the basis of the host cells to be used for expression, which is in operative association with the isolated polynucleotide to be expressed. As used herein with respect to a recombinant expression vector, “in operative association” or “operatively linked” means that the polynucleotide of interest is linked to the regulatory sequence(s) in a manner which allows for expression of the polynucleotide when the vector is introduced into the host cell (e.g., in a bacterial or plant host cell). The term “regulatory sequence” is intended to include promoters, enhancers, and other expression control elements (e.g., polyadenylation signals).

As set forth above, certain embodiments of the invention employ promoters that are capable of enhancing gene expression in leaves. In some embodiments, the promoter is a leaf-specific promoter. Any leaf-specific promoter may be employed in these embodiments of the invention. Many such promoters are known, for example, the USP promoter from Vicia faba (SEQ ID NO: 55 or SEQ ID NO:56, Baeumlein et al. (1991) Mol. Gen. Genet. 225, 459-67), promoters of light-inducible genes such as ribulose-1,5-bisphosphate carboxylase (rbcS promoters), promoters of genes encoding chlorophyll a/b-binding proteins (Cab), Rubisco activase, B-subunit of chloroplast glyceraldehyde 3-phosphate dehydrogenase from A. thaliana, (Kwon et al. (1994) Plant Physiol. 105, 357-67) and other leaf specific promoters such as those identified in Aleman, I. (2001) Isolation and characterization of leaf-specific promoters from alfalfa (Medicago sativa), Masters thesis, New Mexico State University, Los Cruces, N. Mex., and the like a constitutive promoter. Constitutive promoters are active under most conditions. Examples of constitutive promoters suitable for use in these embodiments include the parsley ubiquitin promoter from Petroselinum crispum described in WO 2003/102198 (SEQ ID NO:53); the CaMV 19S and 35S promoters, the sX CaMV 35S promoter, the Sep1 promoter, the rice actin promoter, the A. thaliana actin promoter, the maize ubiquitin promoter, pEmu, the figwort mosaic virus 35S promoter, the Smas promoter, the super promoter (U.S. Pat. No. 5,955,646), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like.

In other embodiments of the invention, a root or shoot specific promoter is employed. For example, the Super promoter (SEQ ID NO: 54) provides high level expression in both root and shoots (Ni et al. (1995) Plant J. 7: 661-676). Other root specific promoters include, without limitation, the TobRB7 promoter (Yamamoto et al. (1991) Plant Cell 3, 371-382), the roID promoter (Leach et al. (1991) Plant Science 79, 69-76); CaMV 35S Domain A (Benfey et al. (1989) Science 244, 174-181), and the like.

In accordance with the invention, a chloroplast transit sequence refers to a nucleotide sequence that encodes a chloroplast transit peptide. Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); ferredoxin NADP+ oxidoreductase (Jansen et al. (1988) Curr. Genetics 13:517-522) (SEQ ID NO:27); nitrite reductase (Back et al (1988) MGG 212:20-26) and the light harvesting chlorophyll a/b binding protein (LHBP) (Lamppa et al. (1988) J. Biol. Chem. 263:14996-14999). See also Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

As defined herein, a mitochondrial transit sequence refers to a nucleotide sequence that encodes a mitochondrial presequence and directs the protein to mitochondria. Examples of mitochondrial presequences include groups consisting of ATPase subunits, ATP synthase subunits, Rieske-FeS protein, Hsp60, malate dehydrogenase, citrate synthase, aconitase, isocitrate dehydrogenase, pyruvate dehydrogenase, malic enzyme, glycine decarboxylase, serine hydroxymethyl transferase, isovaleryl-CoA dehydrogenase and superoxide dismutase. Such transit peptides are known in the art. See, for example, Von Heijne et al. (1991) Plant Mol. Biol. Rep. 9:104-126; Clark et al. (1989) J. Biol. Chem. 264:17544-17550; Romer et al. (1993) Biochem. Biophys. Res. Commun. 196:1414-1421; Faivre-Nitschke et al (2001) Eur J Biochem 268 1332-1339 and Shah et al. (1986) Science 233: 478-481.

In a preferred embodiment of the present invention, the polynucleotides listed in Table 1 are expressed in plant cells from higher plants (e.g., the spermatophytes, such as crop plants). A polynucleotide may be “introduced” into a plant cell by any means, including transfection, transformation or transduction, electroporation, particle bombardment, agroinfection, and the like. Suitable methods for transforming or transfecting plant cells are disclosed, for example, using particle bombardment as set forth in U.S. Pat. Nos. 4,945,050; 5,036,006; 5,100,792; 5,302,523; 5,464,765; 5,120,657; 6,084,154; and the like. More preferably, the transgenic corn seed of the invention may be made using Agrobacterium transformation, as described in U.S. Pat. Nos. 5,591,616; 5,731,179; 5,981,840; 5,990,387; 6,162,965; 6,420,630, U.S. patent application publication number 2002/0104132, and the like. Transformation of soybean can be performed using for example any of the techniques described in European Patent No. EP 0424047, U.S. Pat. No. 5,322,783, European Patent No. EP 0397 687, U.S. Pat. No. 5,376,543, or U.S. Pat. No. 5,169,770. A specific example of wheat transformation can be found in PCT Application No. WO 93/07256. Cotton may be transformed using methods disclosed in U.S. Pat. Nos. 5,004,863; 5,159,135; 5,846,797, and the like. Rice may be transformed using methods disclosed in U.S. Pat. Nos. 4,666,844; 5,350,688; 6,153,813; 6,333,449; 6,288,312; 6,365,807; 6,329,571, and the like. Canola may be transformed, for example, using methods such as those disclosed in U.S. Pat. Nos. 5,188,958; 5,463,174; 5,750,871; EP1566443; WO02/00900; and the like. Other plant transformation methods are disclosed, for example, in U.S. Pat. Nos. 5,932,782; 6,153,811; 6,140,553; 5,969,213; 6,020,539, and the like. Any plant transformation method suitable for inserting a transgene into a particular plant may be used in accordance with the invention.

According to the present invention, the introduced polynucleotide may be maintained in the plant cell stably if it is incorporated into a non-chromosomal autonomous replicon or integrated into the plant chromosomes. Alternatively, the introduced polynucleotide may be present on an extra-chromosomal non-replicating vector and may be transiently expressed or transiently active.

The invention is also embodied in a method of producing a transgenic plant comprising at least one polynucleotide listed in Table 1, wherein expression of the polynucleotide in the plant results in the plant's increased growth and/or yield under normal or water-limited conditions and/or increased tolerance to an environmental stress as compared to a wild type variety of the plant comprising the steps of: (a) introducing into a plant cell an expression cassette described above, (b) regenerating a transgenic plant from the transformed plant cell; and selecting higher-yielding plants from the regenerated plant sells. The plant cell may be, but is not limited to, a protoplast, gamete producing cell, and a cell that regenerates into a whole plant. As used herein, the term “transgenic” refers to any plant, plant cell, callus, plant tissue, or plant part, that contains the expression cassette described above. In accordance with the invention, the expression cassette is stably integrated into a chromosome or stable extra-chromosomal element, so that it is passed on to successive generations.

The effect of the genetic modification on plant growth and/or yield and/or stress tolerance can be assessed by growing the modified plant under normal and/or less than suitable conditions and then analyzing the growth characteristics and/or metabolism of the plant. Such analytical techniques are well known to one skilled in the art, and include measurements of dry weight, wet weight, seed weight, seed number, polypeptide synthesis, carbohydrate synthesis, synthesis, evapotranspiration rates, general plant and/or crop yield, flowering, reproduction, seed setting, root growth, respiration rates, photosynthesis rates, metabolite composition, and the like.

The invention is further illustrated by the following examples, which are not to be construed in any way as imposing limitations upon the scope thereof.

### Example 1

### Characterization of Genes

Nitrogen metabolism genes sII0420 (SEQ ID NO: 1), YPR060C (SEQ ID NO: 19), slr0657 (SEQ ID NO: 23), YMR152W (SEQ ID NO: 31), b2905 (SEQ ID NO: 37), b2066 (SEQ ID NO: 41) and sII0631 (SEQ ID NO: 51) were cloned using standard recombinant techniques. The functionality of each gene was predicted by comparing the amino acid sequence of the encoded protein with other genes of known functionality. Homolog cDNAs were isolated from proprietary libraries of the respective species using known methods. Sequences were processed and annotated using bioinformatics analyses.

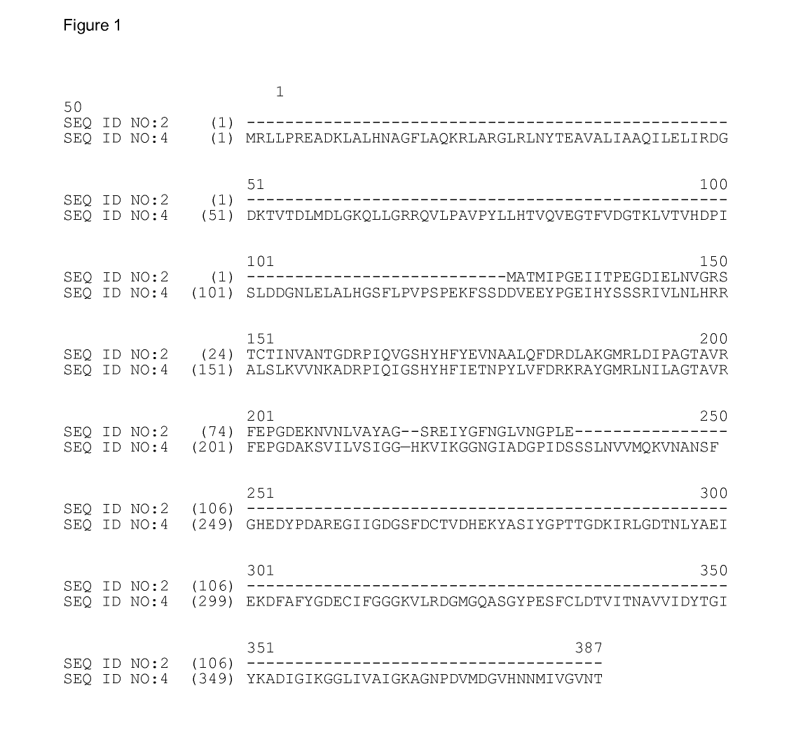

The sII0420 gene (SEQ ID NO: 1) from Synechocystis sp. encodes urease subunit beta. sII0420 (SEQ ID NO: 2). The full-length amino acid sequence of this gene was blasted against proprietary databases of cDNAs at an e value of e⁻¹⁰ (Altschul et al., 1997, Nucleic Acids Res. 25: 3389-3402). One homolog from maize was identified. The amino acid relatedness of these sequences is indicated in the alignments shown in FIG. 1.

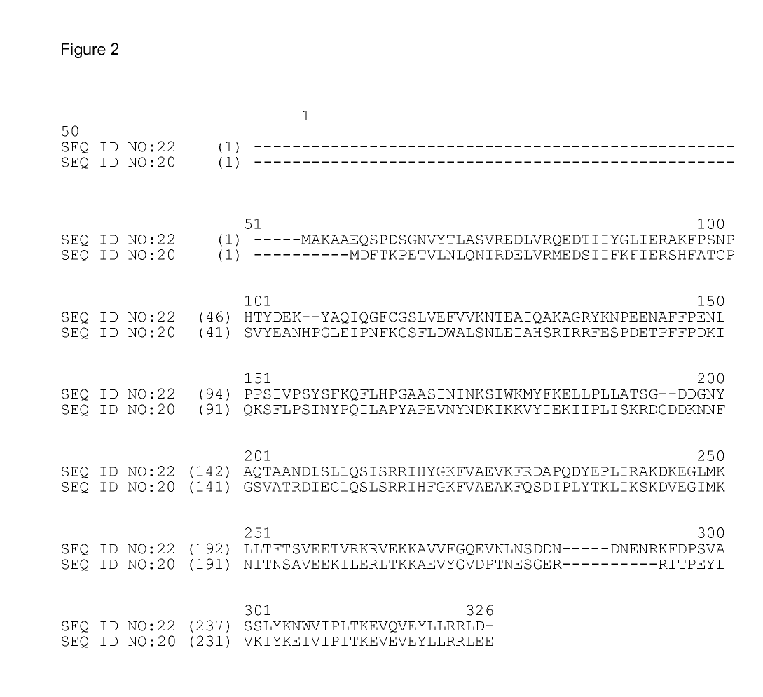

The YPR060C gene (SEQ ID NO: 19) from S. cereviseae encodes chorismate mutase. The full-length amino acid sequence of this gene was blasted against proprietary databases of cDNAs at an e value of e⁻¹⁰ (Altschul et al., supra). One homolog from soybean was identified. The amino acid relatedness of these sequences is indicated in the alignments shown in FIG. 2.

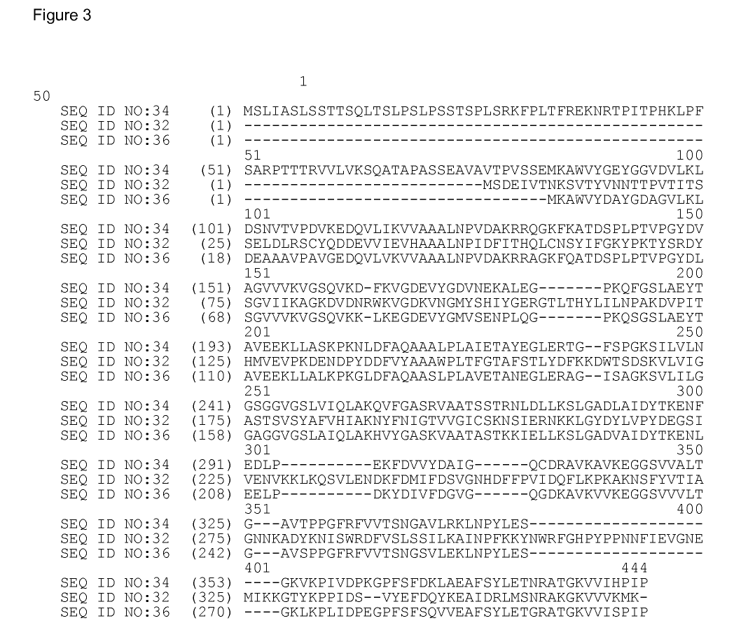

The YMR152W gene (SEQ ID NO: 31) from S. cereviseae encodes a protease similar to an E. coli leader peptidase. The full-length amino acid sequence of this gene was blasted against proprietary databases of cDNAs at an e value of e⁻¹⁰ (Altschul et al., supra). One homolog from soybean and one homolog from maize were identified. The amino acid relatedness of these sequences is indicated in the alignments shown in FIG. 3.

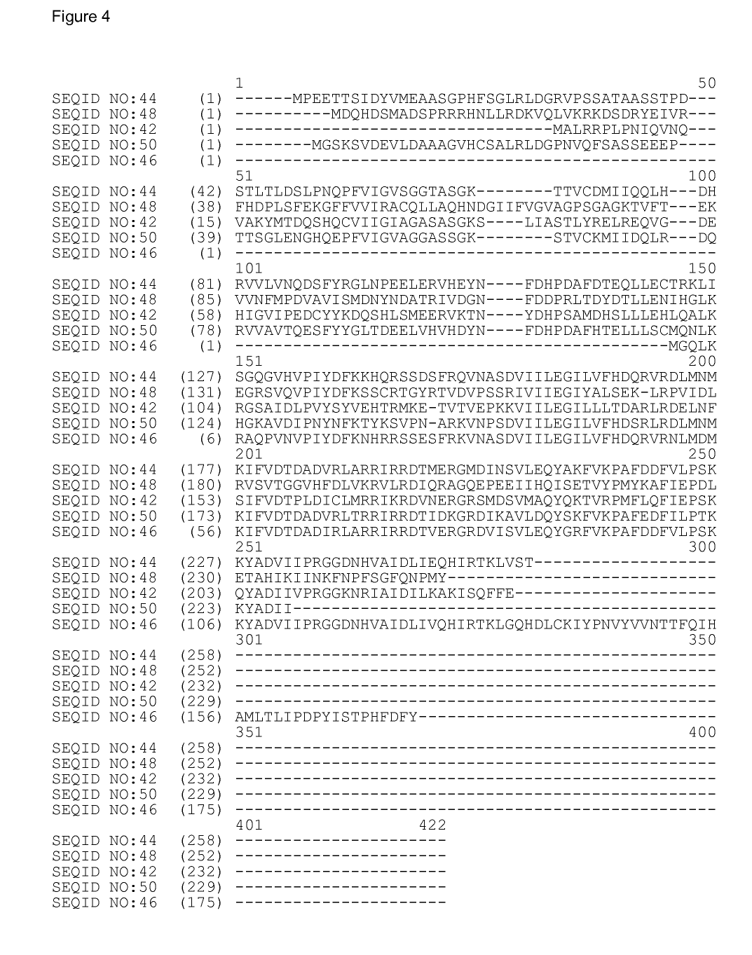

The b2066 gene (SEQ ID NO: 41) from E. coli encodes an aminomethyltransferase. The full-length amino acid sequence of this gene was blasted against proprietary databases of cDNAs at an e value of e⁻¹⁰ (Altschul et al., supra). One homolog from soybean, one from rice, two from maize were identified. The amino acid relatedness of these sequences is indicated in the alignments shown in FIG. 4.

### Example 2

### Overexpression of Lead Genes in Plants

Each of the lead genes described in Example 1 was ligated into an expression cassette using known methods. Three different promoters were used to control expression of the transgenes in A. thaliana described in Tables 3-9: the USP promoter from V. faba (SEQ ID NO: 55 was used for expression of genes from E. coli; SEQ ID NO: 56 for expression of genes from S. cerevisiae); the parsley ubiquitin promoter (SEQ ID NO: 53) designated “PCUbi” for expression of genes from Synechocystis sp; and the super promoter (SEQ ID NO: 54) designated “Super” for expression of genes from E. coli. For targeted expression, the mitochondrial transit peptide from an A. thaliana gene encoding mitochondrial isovaleryl-CoA dehydrogenase (SEQ ID NO: 58, 60) designated “Mito” was used for expression of genes from Synechocystis sp. and E. coli; other genes were expressed with no subcellular targeting.

The A. thaliana ecotype C24 was transformed with constructs containing the lead genes described in Example 1 using known methods. Seeds from T2 transformed plants were pooled on the basis of the promoter driving the expression, gene source species and type of targeting (mitochondrial and cytoplasmic). The seed pools were used in the primary screens for biomass under well watered and water limited growth conditions. Hits from pools in the primary screen were selected, molecular analysis performed and seed collected. The collected seeds were then used for analysis in secondary screens where a larger number of individuals for each transgenic event were analyzed. If plants from a construct were identified in the secondary screen as having increased biomass compared to the controls, it passed to the tertiary screen. In this screen, over 100 plants from all transgenic events for that construct were measured under well watered and drought growth conditions. The data from the transgenic plants were compared to wild type A. thaliana plants or to plants grown from a pool of randomly selected transgenic A. thaliana seeds using standard statistical procedures.

Plants that were grown under well watered conditions were watered to soil saturation twice a week. Images of the transgenic plants were taken at 17 and 21 days using a commercial imaging system. Alternatively, plants were grown under water limited growth conditions by watering to soil saturation infrequently which allowed the soil to dry between watering treatments. In these experiments, water was given on days 0, 8, and 19 after sowing. Images of the transgenic plants were taken at 20 and 27 days using a commercial imaging system.

Image analysis software was used to compare the images of the transgenic and control plants grown in the same experiment. The images were used to determine the relative size or biomass of the plants as pixels and the color of the plants as the ratio of dark green to total area. The latter ratio, termed the health index, was a measure of the relative amount of chlorophyll in the leaves and therefore the relative amount of leaf senescence or yellowing and was recorded at day 27 only. Variation exists among transgenic plants that contain the various lead genes, due to different sites of DNA insertion and other factors that impact the level or pattern of gene expression.

Tables 2 to 8 show the comparison of measurements of the A. thaliana plants. CD indicates that the plants were grown under cycling drought conditions; WW indicates well-watered conditions. Biomass was determined by image analysis at days 20 and 27 after planting when the plants were grown under water limiting conditions and on days 17 and 21 after planting with the well watered treatment. Health Index was measured as the relative amount of dark green in the images. Percent change indicates the measurement of the transgenic relative to the control plants as a percentage of the control non-transgenic plants; p value is the statistical significance of the difference between transgenic and control plants based on a T-test comparison of all independent events; No of events indicates the total number of independent transgenic events tested in the experiment; No of positive events indicates the total number of independent transgenic events that were larger than the control in the experiment; No of negative events indicates the total number of independent transgenic events that were smaller than the control in the experiment. NS indicates not significant at the 5% level of probability.

### A. Urease

Transgenic plants expressing the urease sub unit beta gene sII0420 (SEQ ID NO:1) controlled by the PCUbi promoter with no subcellular targeting were grown under water limiting conditions.

TABLE 2

| | | | | | | | Total # | # of | # of |
|---|---|---|---|---|---|---|---|---|---|
| Assay | | | | | % | p- | of | Pos | Neg |
| Type | Gene | Promoter | Targeting | Measurement | Change | Value | Events | Events | Events |
| CD | ssII0420 | PCUbi | None | Biomass at | 14.2 | 0.001 | 6 | 4 | 2 |
| | | | | day 20 | | | | | |
| CD | ssII0420 | PCUbi | None | Biomass at | 8.5 | 0.028 | 6 | 3 | 3 |
| | | | | day 27 | | | | | |
| CD | ssII0420 | PCUbi | None | Health Index | −3.0 | NS | 6 | 3 | 3 |

Table 2 shows that expression of the sII0420 gene resulted in plants that were larger under water limiting conditions than control plants that did not express the gene. In these experiments, the majority of independent transgenic events expressing the sII0420 gene were larger than the controls indicating better adaptation to the stress environment.

### B. Chorismate Mutase

Transgenic plants expressing the YPR060C gene (SEQ ID NO: 19) controlled by the USP promoter with subcellular targeting to the mitochondria were grown under water limiting or well-watered conditions

TABLE 3

| | | | | | | | Total # | # of | # of |
|---|---|---|---|---|---|---|---|---|---|
| Assay | | | | | % | p- | of | Pos | Neg |
| Type | Gene | Promoter | Targeting | Measurement | Change | Value | Events | Events | Events |
| CD | YPR060C | USP | Mito | Biomass at | 20.2 | 0.000 | 6 | 5 | 1 |
| | | | | day 20 | | | | | |
| CD | YPR060C | USP | Mito | Biomass at | 17.6 | 0.000 | 6 | 6 | 0 |
| | | | | day 27 | | | | | |
| CD | YPR060C | USP | Mito | Health Index | 4.4 | NS | 6 | 5 | 1 |
| WW | YPR060C | USP | Mito | Biomass at | 11.3 | 0.001 | 6 | 4 | 2 |
| | | | | day 17 | | | | | |
| WW | YPR060C | USP | Mito | Biomass at | 5.9 | 0.040 | 6 | 4 | 2 |
| | | | | day 21 | | | | | |
| WW | YPR060C | USP | Mito | Health Index | 2.1 | NS | 6 | 3 | 3 |

Table 3 shows that expression of the YPR060C gene resulted in plants that were larger under water limiting conditions than control plants that did not express the gene. In these experiments, the majority of independent transgenic events expressing the YPR060C gene were larger than the controls indicating better adaptation to the stress environment. Under well watered conditions, expression of the YPR060C gene resulted in plants that were larger than the control plants that did not express YPR060C but the difference to the control was greater under water limiting conditions.

### C. Aspartate Kinase

Transgenic plants expressing the slr0657 gene (SEQ ID NO: 23) controlled by the PCUbi promoter with no subcellular targeting were grown under well-watered conditions.

TABLE 4

| | | | | | | | Total # | # of | # of |
|---|---|---|---|---|---|---|---|---|---|
| Assay | | | | | % | p- | of | Pos | Neg |
| Type | Gene | Promoter | Targeting | Measurement | Change | Value | Events | Events | Events |
| WW | sIr0657 | PCUbi | none | Biomass at | 15.0 | 0.000 | 5 | 4 | 1 |
| | | | | day 17 | | | | | |
| WW | sIr0657 | PCUbi | none | Biomass at | 12.2 | 0.000 | 5 | 4 | 1 |
| | | | | day 21 | | | | | |
| WW | sIr0657 | PCUbi | none | Health Index | −5.7 | 0.030 | 5 | 1 | 4 |

Table 4 shows that expression of the slr0657 gene resulted in plants that were larger under well-watered conditions than control plants that did not express the gene. In these experiments, the majority of independent transgenic events expressing the slr0657 gene were larger than the controls indicating better plant growth.

### D. Protease

Transgenic plants expressing the YMR152W gene (SEQ ID NO:31) controlled by the USP promoter with subcellular targeting to the mitochondria were grown under cycling drought conditions.

TABLE 5

| | | | | | | | Total # | # of | # of |
|---|---|---|---|---|---|---|---|---|---|
| Assay | | | | | % | p- | of | Pos | Neg |
| Type | Gene | Promoter | Targeting | Measurement | Change | Value | Events | Events | Events |
| CD | YMR152W | USP | Mito | Biomass at | 12.4 | 0.002 | 6 | 6 | 0 |
| | | | | day 20 | | | | | |
| CD | YMR152W | USP | Mito | Biomass at | 11.1 | 0.007 | 6 | 4 | 2 |
| | | | | day 27 | | | | | |
| CD | YMR152W | USP | Mito | Health Index | 13.1 | 0.000 | 6 | 5 | 1 |

Table 5 shows that expression of the YMR152W gene resulted in plants that were larger under water limiting conditions than control plants that did not express the gene. In these experiments, the majority of independent transgenic events expressing the YMR152W gene were larger than the controls indicating better adaptation to the stress environment. In addition, the transgenic plants expressing YMR152W were darker green in color than the controls under water limiting conditions as shown by the increased health index. This indicates that the plants produced more chlorophyll or had less chlorophyll degradation during stress than the control plants.

### E. Aminomethyltransferase

Transgenic plants expressing the b2905 gene (SEQ ID NO: 37) controlled by the Super promoter with no subcellular targeting were grown under water limiting or well-watered conditions.

TABLE 6

| | | | | | | | Total # | # of | # of |
|---|---|---|---|---|---|---|---|---|---|
| Assay | | | | | % | p- | of | Pos | Neg |
| Type | Gene | Promoter | Targeting | Measurement | Change | Value | Events | Events | Events |
| CD | b2905 | Super | None | Biomass at | 9.2 | 0.001 | 6 | 5 | 1 |
| | | | | day 20 | | | | | |
| CD | b2905 | Super | None | Biomass at | 14.0 | 0.000 | 6 | 5 | 1 |
| | | | | day 27 | | | | | |
| CD | b2905 | Super | None | Health Index | 0.0 | NS | 6 | 3 | 3 |
| WW | b2905 | Super | None | Biomass at | −3.6 | NS | 6 | 3 | 3 |
| | | | | day 17 | | | | | |
| WW | b2905 | Super | None | Biomass at | −3.5 | NS | 6 | 3 | 3 |
| | | | | day 21 | | | | | |
| WW | b2905 | Super | None | Health Index | −0.8 | NS | 6 | 4 | 2 |

Table 6 shows that expression of the b2905 gene resulted in plants that were larger under water limiting conditions than control plants that did not express the gene. In these experiments, the majority of independent transgenic events expressing the b2905 gene were larger than the controls indicating better adaptation to the stress environment. Under well watered conditions, expression of the b2905 gene resulted in plants that were not significantly different in biomass to the control plants that did not express b2905.

### F. Uridine/Cytidine Kinase

Transgenic plants expressing gene b2066 (SEQ ID NO:41) controlled by the Super or the USP promoter with sub cellular targeting to the mitochondria were grown under water limiting conditions.

TABLE 7

| | | | | | | | Total # | # of | # of |
|---|---|---|---|---|---|---|---|---|---|
| Assay | | | | | % | p- | of | Pos | Neg |
| Type | Gene | Promoter | Targeting | Measurement | Change | Value | Events | Events | Events |
| CD | b2066 | Super | Mito | Biomass at | −4.1 | 0.276 | 6 | 3 | 3 |
| | | | | day 20 | | | | | |
| CD | b2066 | Super | Mito | Biomass at | −11.7 | 0.003 | 6 | 1 | 5 |
| | | | | day 27 | | | | | |
| CD | b2066 | Super | Mito | Health Index | 3.1 | 0.409 | 6 | 4 | 2 |
| CD | b2066 | USP | Mito | Biomass at | 15.3 | 0.000 | 7 | 6 | 1 |
| | | | | day 20 | | | | | |
| CD | b2066 | USP | Mito | Biomass at | 9.6 | 0.003 | 7 | 5 | 2 |
| | | | | day 27 | | | | | |
| CD | b2066 | USP | Mito | Health Index | 9.7 | 0.005 | 7 | 7 | 0 |

Table 7 shows that transgenic plants expressing the b2066 gene controlled by the USP promoter with targeting to the mitochondria were significantly larger under water-limiting conditions than the control plants that did not express the b2066 gene, indicating better adaptation to the stress environment. In addition, the transgenic plants expressing b2066 controlled by the USP promoter were darker green in color than the controls under water limiting conditions as shown by the increased health index. This indicates that the plants produced more chlorophyll or had less chlorophyll degradation during stress than the control plants. When gene b2066 was targeted to the mitochondria under the Super promoter, transgenic plants were smaller than control plants under water-limiting conditions by the end of the cycling-drought assay (day 27).

### G. L-Aspartate Oxidase

Transgenic plants expressing the sII0631 gene (SEQ ID NO:51) controlled by the PCUbi promoter with sub cellular targeting to the mitochondria or no sub cellular targeting were grown under water limiting or well-watered conditions.

TABLE 8

| | | | | | | | Total # | # of | # of |
|---|---|---|---|---|---|---|---|---|---|
| Assay | | | | | % | p- | of | Pos | Neg |
| Type | Gene | Promoter | Targeting | Measurement | Change | Value | Events | Events | Events |
| CD | sII0631 | PCUbi | none | Biomass at | 53.3 | 0.000 | 6 | 6 | 0 |
| | | | | day 20 | | | | | |
| CD | sII0631 | PCUbi | none | Biomass at | 16.8 | 0.000 | 6 | 6 | 0 |
| | | | | day 27 | | | | | |
| CD | sII0631 | PCUbi | none | Health Index | 34.2 | 0.000 | 6 | 6 | 0 |
| WW | sII0631 | PCUbi | Mito | Biomass at | 22.0 | 0.000 | 6 | 6 | 0 |
| | | | | day 17 | | | | | |
| WW | sII0631 | PCUbi | Mito | Biomass at | 10.1 | 0.000 | 6 | 6 | 0 |
| | | | | day 21 | | | | | |
| WW | sII0631 | PCUbi | Mito | Health Index | 22.8 | 0.000 | 6 | 6 | 0 |

Table 8 shows that transgenic plants expressing the sII0631 gene controlled by the PCUbi promoter were significantly larger than control plants that did not express the sII0631 gene under water-limiting conditions. This effect was seen in transgenic plants with no sub cellular targeting under the stress of water-limiting conditions. In addition, the transgenic plants expressing sII0631 were darker green in color than the controls as shown by the increased health index, indicating that the plants produced more chlorophyll or had less chlorophyll degradation during stress than the control plants.

Under well-watered conditions, transgenic plants expressing the sII0631 gene with PCUbi controlled targeting to the mitochondria were larger than control plants not expressing he sII0631 gene. In addition, the transgenic plants expressing sII0631 were darker green in color than the controls as shown by the increased health index. This indicates that the plants produced more chlorophyll or had less chlorophyll degradation under well-watered conditions than the control plants.

1-8. (canceled)

9. A transgenic plant transformed with an expression cassette comprising, in operative association,
a) an isolated polynucleotide encoding a promoter and
b) an isolated polynucleotide encoding a full-length polypeptide comprising a urease beta subunit domain selected from the group consisting of amino acid 5 to amino acid 104 of SEQ ID NO:2; amino acid 132 to amino acid 231 of SEQ ID NO:4; amino acid 133 to amino acid 232 of SEQ ID NO:6; amino acid 2 to amino acid 101 of SEQ ID NO:8; amino acid 2 to amino acid 101 of SEQ ID NO:10; amino acid 2 to amino acid 101 of SEQ ID NO:12; amino acid 2 to amino acid 101 of SEQ ID NO:14; amino acid 133 to amino acid 232 of SEQ ID NO:16; and amino acid 2 to amino acid 101 of SEQ ID NO:18,
wherein the transgenic plant demonstrates increased yield as compared to a wild type plant of the same variety which does not comprise the expression cassette.

10. The transgenic plant of claim 9, wherein the full-length urease polypeptide has a sequence comprising amino acids 1 to 105 of SEQ ID NO: 2; amino acid 1 to amino acid 1 to 385 of SEQ ID NO:4; amino acid 1 to amino acid 838 of SEQ ID NO:6; amino acid 1 to amino acid 101 of SEQ ID NO:8; amino acid 1 to amino acid 124 of SEQ ID NO:10; amino acid 1 to amino acid 101 of SEQ ID NO:12; amino acid 1 to amino acid 101 of SEQ ID NO:14; amino acid 1 to amino acid 837 of SEQ ID NO:16; or amino acid 1 to amino acid 162 of SEQ ID NO:18.

11. A seed is true-breeding for an expression cassette comprising, in operative association,
a) an isolated polynucleotide encoding a promoter and
b) an isolated polynucleotide encoding a full-length polypeptide comprising a urease beta subunit domain selected from the group consisting of amino acid 5 to amino acid 104 of SEQ ID NO:2; amino acid 132 to amino acid 231 of SEQ ID NO:4; amino acid 133 to amino acid 232 of SEQ ID NO:6; amino acid 2 to amino acid 101 of SEQ ID NO:8; amino acid 2 to amino acid 101 of SEQ ID NO:10; amino acid 2 to amino acid 101 of SEQ ID NO:12; amino acid 2 to amino acid 101 of SEQ ID NO:14; amino acid 133 to amino acid 232 of SEQ ID NO:16; and amino acid 2 to amino acid 101 of SEQ ID NO:18,
wherein a transgenic plant grown from said seed demonstrates increased yield as compared to a wild type plant of the same variety which does not comprise the expression cassette.

12. The seed of claim 11, wherein the full-length urease polypeptide has a sequence comprising amino acids 1 to 105 of SEQ ID NO: 2; amino acid 1 to amino acid 385 of SEQ ID NO:4; amino acid 1 to amino acid 386 of SEQ ID NO:6; amino acid 1 to amino acid 101 of SEQ ID NO:8; amino acid 1 to amino acid 124 of SEQ ID NO:10; amino acid 1 to amino acid 101 of SEQ ID NO:12; amino acid 1 to amino acid 101 of SEQ ID NO:14; amino acid 1 to amino acid 837 of SEQ ID NO:16; or amino acid 1 to amino acid 162 of SEQ ID NO:18.

13. A method of increasing yield of a plant, the method comprising the steps of:
a) transforming a wild-type plant cell with an expression cassette comprising, in operative association,
i) an isolated polynucleotide encoding a promoter and
ii) an isolated polynucleotide encoding a full-length polypeptide comprising a urease beta domain selected from the group consisting of amino acid 5 to amino acid 104 of SEQ ID NO:2; amino acid 132 to amino acid 231 of SEQ ID NO:4; amino acid 133 to amino acid 232 of SEQ ID NO:6; amino acid 2 to amino acid 101 of SEQ ID NO:8; amino acid 2 to amino acid 101 of SEQ ID NO:10; amino acid 2 to amino acid 101 of SEQ ID NO:12; amino acid 2 to amino acid 101 of SEQ ID NO:14; amino acid 133 to amino acid 232 of SEQ ID NO:16; and amino acid 2 to amino acid 101 of SEQ ID NO:18;
b) regenerating transgenic plants from the transformed plant cell; and
c) selecting higher-yielding plants from the regenerated plants.

14. The method of claim 13, wherein the full-length urease polypeptide has a sequence comprising amino acids 1 to 105 of SEQ ID NO: 2; amino acid 1 to amino acid 385 of SEQ ID NO:4; amino acid 1 to amino acid 386 of SEQ ID NO:6; amino acid 1 to amino acid 101 of SEQ ID NO:8; amino acid 1 to amino acid 124 of SEQ ID NO:10; amino acid 1 to amino acid 101 of SEQ ID NO:12; amino acid 1 to amino acid 101 of SEQ ID NO:14; amino acid 1 to amino acid 837 of SEQ ID NO:16; or amino acid 1 to amino acid 162 of SEQ ID NO:18.


SEQUENCE LISTING (60 sequences)

SEQ ID NO: 1 - DNA, 318 bp, Synechocystis sp. PCC 6803
  CDS (1)..(318): Urease (SLL0420)
atggcaactatgattcctggcgaaatcattacccccgaaggtgacatagaactaaacgtaggccgctccacctgcactattaatgtggccaacactggcgatcgccctattcaagtgggttcccattaccatttctacgaagtcaatgccgctctgcaattcgaccgggatttggccaaggggatgcggttggatattccagcgggaacggcggtgcgctttgagccaggggacgaaaaaaacgttaacttagtggcctatgctggcagtcgggaaatctatggcttcaatggcttggtgaacggccctctagagtga

SEQ ID NO: 2 - protein, 105 aa, Synechocystis sp. PCC 6803
MATMIPGEIITPEGDIELNVGRSTCTINVANTGDRPIQVGSHYHFYEVNAALQFDRDLAKGMRLDIPAGTAVRFEPGDEKNVNLVAYAGSREIYGFNGLVNGPLE

SEQ ID NO: 3 - DNA, 1155 bp, Zea mays
  CDS (1)..(1155): Urease (ZM07mc17684)
atgaggctgctgccgagggaggcggacaaattggcgctgcacaatgccggcttcctcgcgcagaagcgccttgcccggggcctccgcctcaactacaccgaggccgtcgcgctcatcgccgcgcagattcttgagcttattcgcgatggagacaaaaccgtgacagacctcatggacctggggaaacaactattgggcaggagacaagttcttccagctgttccataccttttacatactgtgcaggttgaaggaacatttgtggatggaacaaaactagttactgtacacgaccctatctccttggatgacggaaatttggagctagcattgcatggttcttttctcccagtgccttcacctgaaaagttttccagcgatgacgttgaagaatatcctggtgaaatacattacagttctagtcgcatagtcctaaaccttcatcgcagggctttatctctgaaggttgttaacaaggcagacagacccattcagattggaagccattaccattttatagagaccaatccttatctggtttttgataggaaaagggcctatggcatgaggttgaacatacttgctggaacagctgttcggtttgagccaggggatgcaaaaagtgttatacttgtaagcatcggaggtcataaggtaatcaaaggtggaaatggcattgctgatggtcctattgacagttcaagtcttaatgtggtgatgcagaaggttaatgcaaatagttttggacatgaagattatccagatgcaagggaaggtattattggtgatggttcatttgactgtactgttgatcatgagaagtatgcgagcatttatggacctaccaccggtgataagattaggcttggtgataccaatctttatgctgagattgaaaaggacttcgccttttatggtgatgagtgcatatttggcggtggaaaagttctgcgtgatggcatgggacaagcttcagggtacccagaatctttctgcctagatacagttataacaaatgctgttgtgattgattataccggaatatacaaggctgatatcggtataaaaggtggacttatagttgctattggaaaggctggaaaccctgatgtcatggatggtgtccataacaacatgattgttggggtcaatact

SEQ ID NO: 4 - protein, 385 aa, Zea mays
MRLLPREADKLALHNAGFLAQKRLARGLRLNYTEAVALIAAQILELIRDGDKTVTDLMDLGKQLLGRRQVLPAVPYLLHTVQVEGTFVDGTKLVTVHDPISLDDGNLELALHGSFLPVPSPEKFSSDDVEEYPGEIHYSSSRIVLNLHRRALSLKVVNKADRPIQIGSHYHFIETNPYLVFDRKRAYGMRLNILAGTAVRFEPGDAKSVILVSIGGHKVIKGGNGIADGPIDSSSLNVVMQKVNANSFGHEDYPDAREGIIGDGSFDCTVDHEKYASIYGPTTGDKIRLGDTNLYAEIEKDFAFYGDECIFGGGKVLRDGMGQASGYPESFCLDTVITNAVVIDYTGIYKADIGIKGGLIVAIGKAGNPDVMDGVHNNMIVGVNT

SEQ ID NO: 5 - DNA, 2514 bp, Arabidopsis thaliana
  CDS (1)..(2514): Urease (NP_1766922)
atgaagctgctgccgagggagatcgagaagctggagctgcaccaggccggcttcctggcccagaagaggctggccaggggcatcaggctgaactacaccgaggccgtggccctgatcgccacccagatcctggagttcatcagggacggcgacaagagcgtggccgagctgatggacatcggcaggcagctgctgggcaggaggcaggtgctgccggccgtgctgcacctgctgtacaccgtgcaggtggagggcaccttcagggacggcaccaagctggtgaccgtgcacgagccgatcagcctggagaacggcaacctggagctggccctgcacggcagcttcctgccggtgccgagcctggacaagttcccggaggtgcacgagggcgtgatcatcccgggcgacatgaagtacggcgacggcagcatcatcatcaaccacggcaggaaggccgtggtgctgaaggtggtgaacaccggcgacaggccggtgcaggtgggcagccactaccacttcatcgaggtgaacccgctgctggtgttcgacaggaggaaggccctgggcatgaggctgaacatcccggccggcaccgccgtgaggttcgagccgggcgagaggaagagcgtggtgctggtgaacatcggcggcaacaaggtgatcaggggcggcaacggcatcgtggacggcctggtggacgacgtgaactggaccgtgctgatggagaccatggagaggaggggcttcaagcacctggaggacatcgacgccagcgagggcatcgccggcgaggacccgaggttcaccaccatgatcagcagggagaagtacgccaacatgtacggcccgaccaccggcgacaagctgaggctgggcgacaccaacctgtacgccaggatcgagaaggactacaccgtgtacggcgacgagtgcgtgttcggcggcggcaaggtgctgagggagggcatgggccagggcatcgagcaggccgaggccctgagcctggacaccgtgatcaccaacagcgtgatcatcgactacagcggcatctacaaggccgacatcggcatcaagaacggccacatcgtgggcatcggcaaggccggcaacccggacaccatgcacggcgtgcagaacaacatgctgatcggcaacaagaccgaggtgatcgccggcgagggcatgatcgtgaccgccggcgccatcgactgccacgtgcacttcatctgcccgcagctggtgtacgaggccgtgagcagcggcatcaccaccatggtgggcggcggcaccggcccggcctacggcaccagggccaccacctgcaccccgagcccgttcgacatgaagctgatgctgcagagcaccgacagcctgccgctgaacttcggcttcaccggcaagggcaacaccgccaagccgctggagctgaggcacatcgtggaggccggcgccatgggcctgaagctgcacgaggactggggcaccaccccggccgccatcgacaactgcctggccgtggccgaggagtacgacatccaggtgaacatccacaccgacaccctgaacgagagcggcttcgtggagcacaccatcaacgccttcaggggcaggaccatccacacctaccacagcgagggcgccggcggcggccacgccccggacatcatcagggtgtgcggcgtgaagaacgtgctgccgagcagcaccaacccgaccaggccgtacaccaagaacaccgtggacgagcacctggacatgctgatggtgtgccaccacctggacaagaacatcccggaggacgtggccttcgccgagagcaggatcagggccgagaccatcgccgccgaggacatcctgcacgacatgggcgccatcagcatcatcagcagcgacagccaggccatgggcaggatcggcgaggtgatcagcaggacctggcagaccgccgacaagatgaaggcccagaggggcgccatcgacccgaacatggccgacgacgacaacagcaggatcaagaggtacatcgccaagtacaccatcaacccggccatcgccaacggcttcgccgacctgatcggcagcgtggaggtgaagaagctggccgacctggtgatctggcagccggccttcttcggcgccaagccggagatgatcatcaagggcggcaacatcgcctgggccaacatgggcgacgccaacgccagcatcccgaccccggagccggtgatcagcaggccgatgttcggcgccttcggcaaggccggcagcgagaacagcgtggccttcgtgagcaaggccgccctgaggaagggcgtgaaggagctgtacggcctgaagaagagggtggtggccgtgagcaacgtgaggcagctgaccaagctggacatgaagctgaacgacgccctgccggagatcaccgtggacccggagacctacgtggtgaccgccaacggcgaggtgctgacctgcgccccggccgacagcgtgccgctgagcaggaactacttcctgttc

SEQ ID NO: 6 - protein, 838 aa, Arabidopsis thaliana
MKLLPREIEKLELHQAGFLAQKRLARGIRLNYTEAVALIATQILEFIRDGDKSVAELMDIGRQLLGRRQVLPAVLHLLYTVQVEGTFRDGTKLVTVHEPISLENGNLELALHGSFLPVPSLDKFPEVHEGVIIPGDMKYGDGSIIINHGRKAVVLKVVNTGDRPVQVGSHYHFIEVNPLLVFDRRKALGMRLNIPAGTAVRFEPGERKSVVLVNIGGNKVIRGGNGIVDGLVDDVNWTVLMETMERRGFKHLEDIDASEGIAGEDPRFTTMISREKYANMYGPTTGDKLRLGDTNLYARIEKDYTVYGDECVFGGGKVLREGMGQGIEQAEALSLDTVITNSVIIDYSGIYKADIGIKNGHIVGIGKAGNPDTMHGVQNNMLIGNKTEVIAGEGMIVTAGAIDCHVHFICPQLVYEAVSSGITTMVGGGTGPAYGTRATTCTPSPFDMKLMLQSTDSLPLNFGFTGKGNTAKPLELRHIVEAGAMGLKLHEDWGTTPAAIDNCLAVAEEYDIQVNIHTDTLNESGFVEHTINAFRGRTIHTYHSEGAGGGHAPDIIRVCGVKNVLPSSTNPTRPYTKNTVDEHLDMLMVCHHLDKNIPEDVAFAESRIRAETIAAEDILHDMGAISIISSDSQAMGRIGEVISRTWQTADKMKAQRGAIDPNMADDDNSRIKRYIAKYTINPAIANGFADLIGSVEVKKLADLVIWQPAFFGAKPEMIIKGGNIAWANMGDANASIPTPEPVISRPMFGAFGKAGSENSVAFVSKAALRKGVKELYGLKKRVVAVSNVRQLTKLDMKLNDALPEITVDPETYVVTANGEVLTCAPADSVPLSRNYFLF

SEQ ID NO: 7 - DNA, 303 bp, Sinorhizobium meliloti
  CDS (1)..(303): Urease(CAC47052)
atgatcccgggcgagatcatcgccgccgccggcgagatcgagctgaacgccggcctggagaccgtgagcatcgaggtggccaacagcggcgacaggccggtgcaggtgggcagccactaccacttcgccgagaccaacccgggcctgatcttcgacagggacgccgccaggggcaagaggctggacatcccggccggcaccgccgtgaggttcgagccgggccagaccaggcaggtgaccctgatcccgctgagcggcaagagggaggtgttcggcttcaggcagcaggtgatgggcaagctg

SEQ ID NO: 8 - protein, 101 aa, Sinorhizobium meliloti
MIPGEIIAAAGEIELNAGLETVSIEVANSGDRPVQVGSHYHFAETNPGLIFDRDAARGKRLDIPAGTAVRFEPGQTRQVTLIPLSGKREVFGFRQQVMGKL

SEQ ID NO: 9 - DNA, 372 bp, Bacillus amyloliquefaciens
  CDS (1)..(372)
atgaagccgggcgccatccaggtggccaagggcatcatcaccatcaacgagggcaggaagaccctggaggtgagcgtgaccaacaacggcaccaggagcgtgcaggtgggcagccacttccacttcgccgaggccaacggcgccctgagcttcaacagggacaaggccatcggcatgaggctggacatcccgagcggcaccagcgtgaggttcgagccgggcgaggagaagaccgtgaccctggtggagatcggcggcaggaagaccgtgaggggcctgaacggcatggccgacacctacatggacgagaggggcaaggagaagaccctgagcaacctgaagaaggccggctggatggaggaggccatcagg

SEQ ID NO: 10 - protein, 124 aa, Bacillus amyloliquefaciens
MKPGAIQVAKGIITINEGRKTLEVSVTNNGTRSVQVGSHFHFAEANGALSFNRDKAIGMRLDIPSGTSVRFEPGEEKTVTLVEIGGRKTVRGLNGMADTYMDERGKEKTLSNLKKAGWMEEAIR

SEQ ID NO: 11 - DNA, 303 bp, Agrobacterium tumefaciens
  CDS (1)..(303): Urease (Q8UCS8)
atgaagccgggcgagatcatcgccgccgagggcaccatcgagctgaacgccggccagccgaccgtgaccatcgaggtggccaacagcggcgacaggccggtgcaggtgggcagccactaccacttcttcgagaccaacgccggcctgctgttcgacagggacaaggccaggggcatgaggctggacatcccggccggcaccgccgtgaggttcgagccgggccagaagagggaggtgaccctggtgccgctgagcggcaagagggaggtgtacggcttcaggcagcagatcatgggcaggctg

SEQ ID NO: 12 - protein, 101 aa, Agrobacterium tumefaciens
MKPGEIIAAEGTIELNAGQPTVTIEVANSGDRPVQVGSHYHFFETNAGLLFDRDKARGMRLDIPAGTAVRFEPGQKREVTLVPLSGKREVYGFRQQIMGRL

SEQ ID NO: 13 - DNA, 303 bp, Rhizobium leguminosarum bv.Trifolii
  CDS (1)..(303): Urease (B0IYC4)
atgatcccgggcgagatcttcgccgccagcggcgacatcgagctgaacgccggcgccccgaccgtgaccctggaggtgagcaacaccggcgacaggccggtgcaggtgggcagccactaccacttcgccgagaccaacgccggcctgagcttcgacagggccgccgcccacggcaagaggctggacatcccggccggcaccgccgtgaggttcgagccgggccagaccaggagcgtgaccctgatcccgctgagcggcaagagggaggtgtacggcttcaggcagctggtgatgggcaagctg

SEQ ID NO: 14 - protein, 101 aa, Rhizobium leguminosarum bv.Trifolii
MIPGEIFAASGDIELNAGAPTVTLEVSNTGDRPVQVGSHYHFAETNAGLSFDRAAAHGKRLDIPAGTAVRFEPGQTRSVTLIPLSGKREVYGFRQLVMGKL

SEQ ID NO: 15 - DNA, 2511 bp, Glycine max
  CDS (1)..(2511): Urease (AAO85883)
atgaagctgagcccgagggagatcgagaagctggacctgcacaacgccggctacctggcccagaagaggctggccaggggcctgaggctgaactacgtggagaccgtggccctgatcgccacccagatcctggagttcgtgagggacggcgagaagaccgtggcccagctgatgtgcatcggcagggagctgctgggcaggaagcaggtgctgccggccgtgccgcacctggtggagagcgtgcaggtggaggccaccttcagggacggcaccaagctggtgaccatccacgacctgttcgcctgcgagaacggcaacctggagctggccctgttcggcagcttcctgccggtgccgagcctggacaagttcaccgagaacgaggaggaccacaggaccccgggcgagatcatctgcaggagcgagaacctgatcctgaacccgaggaggaacgccatcatcctgagggtggtgaacaagggcgacaggccgatccaggtgggcagccactaccacttcatcgaggtgaacccgtacctgaccttcgacaggaggaaggcctacggcatgaggctgaacatcgccgccggcaacgccaccaggttcgagccgggcgagtgcaagagcgtggtgctggtgagcatcggcggcaacaaggtgatcaggggcggcaacaacatcgccgacggcccggtgaacgacagcaactgcagggccgccatgaaggccgtggtgaccaggggcttcggccacgtggaggaggagaacgccagggagggcgtgaccggcgaggactacagcctgaccaccgtgatcagcagggaggagtacgcccacaagtacggcccgaccaccggcgacaagatcaggctgggcgacaccgacctgttcgccgagatcgagaaggacttcgccgtgtacggcgacgagtgcgtgttcggcggcggcaaggtgatcagggacggcatgggccagagcagcggccacccgccggagggcagcctggacaccgtgatcaccaacgccgtgatcatcgactacaccggcatcatcaaggccgacatcggcatcaaggacggcctgatcatcagcaccggcaaggccggcaacccggacatcatgaacgacgtgttcccgaacatgatcatcggcgccaacaccgaggtgatcgccggcgagggcctgatcgtgaccgccggcgccatcgactgccacgtgcacttcatctgcccgcagctggtgtacgacgccgtgaccagcggcatcaccaccctggtgggcggcggcaccggcccggccgacggcaccagggccaccacctgcaccccggccccgaaccagatgaagctgatgctgcagagcaccgacgacatgccgctgaacttcggcttcaccggcaagggcaacagcgccaagccggacgagctgcacgagatcatcagggccggcgccatgggcctgaagctgcacgaggactggggcaccaccccggccgccatcgacagctgcctgaccgtggccgaccagtacgacatccaggtgaacatccacaccgacaccctgaacgagagcggcttcgtggagcacaccatcgccgccttcaagggcaggaccatccacacctaccacagcgagggcgccggcggcggccacgccccggacatcatcaaggtgtgcggcgagaagaacgtgctgccgagcagcaccaacccgaccaggccgtacacccacaacaccatcgacgagcacctggacatgctgatggtgtgccaccacctgaacaagaacatcccggaggacgtggccttcgccgagagcaggatcagggccgagaccatcgccgccgaggacatcctgcacgacaagggcgccatcagcatcatcagcagcgacagccaggccatgggcaggatcggcgaggtgatcagcaggacctggcagaccgccgacaagatgaagagccagaggggcccgctgcagccgggcgaggacaacgacaacttcaggatcaagaggtacgtggccaagtacaccatcaacccggccatcgccaacggcctgagccagtacgtgggcagcgtggaggccggcaagctggccgacctggtgctgtggaagccgagcttcttcggcgccaagccggagatggtgatcaagggcggcgaggtggcctacgccaacatgggcgacccgaacgccagcatcccgaccccggagccggtgatcatgaggccgatgttcggcgccttcggcaaggccggcagcagccacagcatcgccttcgtgagcaaggccgccctggacgagggcgtgaaggccagctacggcctgaacaagagggtggaggccgtgaagaacgtgaggaagctgaccaagagggacatgaagctgaacgacaccctgccgcagatcaccgtggacccggagacctacaccgtgaccgccgacggcgaggtgctgacctgcaccgccgccaagaccgtgccgctgagcaggaactacttcctgttc

SEQ ID NO: 16 - protein, 837 aa, Glycine max
MKLSPREIEKLDLHNAGYLAQKRLARGLRLNYVETVALIATQILEFVRDGEKTVAQLMCIGRELLGRKQVLPAVPHLVESVQVEATFRDGTKLVTIHDLFACENGNLELALFGSFLPVPSLDKFTENEEDHRTPGEIICRSENLILNPRRNAIILRVVNKGDRPIQVGSHYHFIEVNPYLTFDRRKAYGMRLNIAAGNATRFEPGECKSVVLVSIGGNKVIRGGNNIADGPVNDSNCRAAMKAVVTRGFGHVEEENAREGVTGEDYSLTTVISREEYAHKYGPTTGDKIRLGDTDLFAEIEKDFAVYGDECVFGGGKVIRDGMGQSSGHPPEGSLDTVITNAVIIDYTGIIKADIGIKDGLIISTGKAGNPDIMNDVFPNMIIGANTEVIAGEGLIVTAGAIDCHVHFICPQLVYDAVTSGITTLVGGGTGPADGTRATTCTPAPNQMKLMLQSTDDMPLNFGFTGKGNSAKPDELHEIIRAGAMGLKLHEDWGTTPAAIDSCLTVADQYDIQVNIHTDTLNESGFVEHTIAAFKGRTIHTYHSEGAGGGHAPDIIKVCGEKNVLPSSTNPTRPYTHNTIDEHLDMLMVCHHLNKNIPEDVAFAESRIRAETIAAEDILHDKGAISIISSDSQAMGRIGEVISRTWQTADKMKSQRGPLQPGEDNDNFRIKRYVAKYTINPAIANGLSQYVGSVEAGKLADLVLWKPSFFGAKPEMVIKGGEVAYANMGDPNASIPTPEPVIMRPMFGAFGKAGSSHSIAFVSKAALDEGVKASYGLNKRVEAVKNVRKLTKRDMKLNDTLPQITVDPETYTVTADGEVLTCTAAKTVPLSRNYFLF

SEQ ID NO: 17 - DNA, 486 bp, Corynebacterium glutamicum
  CDS (1)..(486): Urease (NP599337)
atgatcccgggcgagtacatcctgagcagcgagagcctgaccggcaacgtgggcagggaggccaagaccatcgagatcatcaacaccggcgacaggccggtgcagatcggcagccacttccacttcgccgaggtgaacccgagcatcagcttcgacaggagcgagggctacggcttcaggctggacatcccgagcggcaccgccgtgaggctggagccgggcgacgccaggaccgtgaacctggtggccatcggcggcgacaggatcgtggccggcttcagggacctggtggacggcccgctggaggacctgaaggtgaacgtgtgggagggcagggaggacggctggaggaggagcagcgccgccggcgacgccccgcaggagctgccgcaggtggaggccgccgagaggggcaggaagctggacgacgccaccgacgtggacaccaacgtgggcaccgaggagggcttcgaggagggcaggaac

SEQ ID NO: 18 - protein, 162 aa, Corynebacterium glutamicum
MIPGEYILSSESLTGNVGREAKTIEIINTGDRPVQIGSHFHFAEVNPSISFDRSEGYGFRLDIPSGTAVRLEPGDARTVNLVAIGGDRIVAGFRDLVDGPLEDLKVNVWEGREDGWRRSSAAGDAPQELPQVEAAERGRKLDDATDVDTNVGTEEGFEEGRN

SEQ ID NO: 19 - DNA, 771 bp, Sacharomyces cereviseae
  CDS (1)..(771): Chorismate Mutase (ypr060c)
atggatttcacaaaaccagaaactgttttaaatctacaaaatattagagatgaattagttagaatggaggattcgatcatcttcaaatttattgagaggtcgcatttcgccacatgtccttcagtttatgaggcaaaccatccaggtttagaaattccgaattttaaaggatctttcttggattgggctctttcaaatcttgaaattgcgcattctcgcatcagaagattcgaatcacctgatgaaactcccttctttcctgacaagattcagaaatcattcttaccgagcattaactacccacaaattttggcgccttatgccccagaagttaattacaatgataaaataaaaaaagtttatattgaaaagattataccattaatttcgaaaagagatggtgatgataagaataacttcggttctgttgccactagagatatagaatgtttgcaaagcttgagtaggagaatccactttggcaagtttgttgctgaagccaagttccaatcggatatcccgctatacacaaagctgatcaaaagtaaagatgtcgaggggataatgaagaatatcaccaattctgccgttgaagaaaagattctagaaagattaactaagaaggctgaagtctatggtgtggaccctaccaacgagtcaggtgaaagaaggattactccagaatatttggtaaaaatttataaggaaattgttatacctatcactaaggaagttgaggtggaatacttgctaagaaggttggaagagtaa

SEQ ID NO: 20 - protein, 256 aa, Sacharomyces cereviseae
MDFTKPETVLNLQNIRDELVRMEDSIIFKFIERSHFATCPSVYEANHPGLEIPNFKGSFLDWALSNLEIAHSRIRRFESPDETPFFPDKIQKSFLPSINYPQILAPYAPEVNYNDKIKKVYIEKIIPLISKRDGDDKNNFGSVATRDIECLQSLSRRIHFGKFVAEAKFQSDIPLYTKLIKSKDVEGIMKNITNSAVEEKILERLTKKAEVYGVDPTNESGERRITPEYLVKIYKEIVIPITKEVEVEYLLRRLEE

SEQ ID NO: 21 - DNA, 786 bp, Glycine max
  CDS (1)..(786): Chorismate Mutase (GM06MC36749)
atggccaaagcagcagaacaaagtcctgattctgggaatgtgtacacgctagcttctgtgagagaggatttggttaggcaagaggataccatcatttatggtctcattgagagagccaagttccctagcaattctcacacctatgatgaaaagtatgctcaaatccagggtttttgtggctcattggtggaatttgttgttaagaatacagaggccattcaagctaaggctggaagatacaaaaaccctgaagaaaacgccttcttcccagaaaatttaccaccatcaattgtgccatcttactccttcaaacagtttttgcatcctggtgctgcttcaattaacataaacaagtccatctggaaaatgtatttcaaagagttacttccattgcttgctacttcgggtgatgatggcaactatgcgcaaactgcagctaatgacctttcattattgcagtccatctctagaaggattcactatggaaagtttgtagctgaggtgaaattcagggatgctcctcaagactacgagcctttaattcgagctaaggataaagaaggattgatgaaattgttgacatttacaagcgttgaagagacggtgaggaagagagttgaaaagaaggctgtggtgtttgggcaggaagtgaatcttaacagtgatgacaatgacaatgaaaaccgtaaatttgatccatcagtggcttctagcttgtacaaaaattgggtgatacctctcaccaaggaggttcaggttgagtacctcttgcgccgtctagactga

SEQ ID NO: 22 - protein, 261 aa, Glycine max
MAKAAEQSPDSGNVYTLASVREDLVRQEDTIIYGLIERAKFPSNSHTYDEKYAQIQGFCGSLVEFVVKNTEAIQAKAGRYKNPEENAFFPENLPPSIVPSYSFKQFLHPGAASININKSIWKMYFKELLPLLATSGDDGNYAQTAANDLSLLQSISRRIHYGKFVAEVKFRDAPQDYEPLIRAKDKEGLMKLLTFTSVEETVRKRVEKKAVVFGQEVNLNSDDNDNENRKFDPSVASSLYKNWVIPLTKEVQVEYLLRRLD

SEQ ID NO: 23 - DNA, 1803 bp, Synechocystis sp. PCC 6803
  CDS (1)..(1803): Aspartate Kinase (slr0657)
atggcattaatcgttcaaaaatttggcggcacttccgttggcactgtggagcgtattcaagcggtggcccaacgcattaaacgaactgtgcagggaggcaactccctggtggtggtggtgtcggccatgggaaaaagcaccgatgtgttagtggatttagcccaacagatttcccccaatccctgtcgccgggagatggatatgttactttccaccggggaacaggtatccattgctttgctgagtttagccttgcaggaaatagaccagcccgccatttccctcaccggagcccaggtgggcatcgtcaccgaggccgaacatagccgggcccgcattctagaaattcgtcccgatcgcctggagcaccatttaagggaagggaaagtggtggtagtggctggtttccagggcatcagtagcgtagaacatctagaaattactaccctgggacggggcggctcagacacctctgccgtggccctggcggcggcattgaaagcggatttttgcgaaatttacaccgacgtaccgggtattctcaccactgatccccgtttagtgccggaagcccaactgatggcggaaatcacctgtgacgagatgttggaattagccagtctgggggcgaaagtactccatccccgggcggtggaaattgcccgcaattatggcattcccttggtggtgcgttccagttggagtgatgaaccgggcacaaaagtagtggctcccccggtgcaaaaccgttccctggtagggcttgaaattgccaaggcagtggatggggtggaatacgacgccgatcaagccaaagtcgctctgttgcgagtgcccgatcgccctggggtggcatcaaaattatttcgggatattgcccaacaacaggtggacattgatttaattattcaatccatccatgacggcaacagtaatgacattgctttcactgttgtcaaagatttactcaatacagcggaagccgttaccagcgccattgccccggccctacgcagttaccccgaagcggatcaggaagcggaaattattgtcgaaaaaggcatcgctaaaattgccattgccggagccggtatgatcggtcgtcctggcatcgccgccaaaatgttcaaaactttggccgatgtgggggtcaacattgaaatgatttccacttcggaagtgaaagttagttgtgttattgatcaacgggacgcagaccgggcgatcgccgctttatccaatgcttttggggtaactttatctccgccgaaaaaccaaacggacacttcccatttaccagcggtgcggggggtagccctggaccaagaccaggcccaaattgccattcgccacgtacccgatcgccctggcatggcagcccaactgttcaccgccctagcagaagccaacatcagtgtggacatgattatccaatcccaacgctgtcgcattaatcaaggcaccccctgccgggacattgcttttatggtggcggaaggggatagcagtcaggctgaggcgattttgcaacccctgatcaaagattggctcgatgcggccatagttgtgaacaaagccattgccaaagtcagcattgtcggttccggcatgattggccatccgggggtggcggcccacttctttgctgccctggcccaggaaaatatcaacattgaaatgattgccacgtcggaaatcaaaatcagttgtgtggtgccccaggatcggggagtggatgctttgaaagctgcccacagcgcctttaacttagctggcacgaaaaccgtcactgttccagcttag

SEQ ID NO: 24 - protein, 600 aa, Synechocystis sp. PCC 6803
MALIVQKFGGTSVGTVERIQAVAQRIKRTVQGGNSLVVVVSAMGKSTDVLVDLAQQISPNPCRREMDMLLSTGEQVSIALLSLALQEIDQPAISLTGAQVGIVTEAEHSRARILEIRPDRLEHHLREGKVVVVAGFQGISSVEHLEITTLGRGGSDTSAVALAAALKADFCEIYTDVPGILTTDPRLVPEAQLMAEITCDEMLELASLGAKVLHPRAVEIARNYGIPLVVRSSWSDEPGTKVVAPPVQNRSLVGLEIAKAVDGVEYDADQAKVALLRVPDRPGVASKLFRDIAQQQVDIDLIIQSIHDGNSNDIAFTVVKDLLNTAEAVTSAIAPALRSYPEADQEAEIIVEKGIAKIAIAGAGMIGRPGIAAKMFKTLADVGVNIEMISTSEVKVSCVIDQRDADRAIAALSNAFGVTLSPPKNQTDTSHLPAVRGVALDQDQAQIAIRHVPDRPGMAAQLFTALAEANISVDMIIQSQRCRINQGTPCRDIAFMVAEGDSSQAEAILQPLIKDWLDAAIVVNKAIAKVSIVGSGMIGHPGVAAHFFAALAQENINIEMIATSEIKISCVVPQDRGVDALKAAHSAFNLAGTKTVTVPA

SEQ ID NO: 25 - DNA, 1584 bp, Saccharomyces cerevisiae
  CDS (1)..(1584): Aspartate Kinase (hom3)
atgccaatggatttccaacctacatcaagtcattcgaactgggtcgtgcaaaagttcggtggtacatctgtcggtaaatttcccgtccaaatagtggatgacattgtgaagcactattctaaacctgacggcccaaacaataatgtcgctgtcgtttgttccgcccgttcttcatacaccaaggctgaaggtaccacttctcgtcttttgaaatgttgtgatttggcttcgcaagaatctgaatttcaagacattatcgaagttatcagacaagaccatatcgataatgccgaccgcttcattctcaatcctgccttgcaagccaagttagtggatgataccaataaagaacttgaactggtcaagaaatatttaaatgcttcaaaagttttgggtgaagtgagttcacgtacagtagatctggtgatgtcatgtggtgagaagttgagttgtttgttcatgactgctttatgtaatgaccgtggctgtaaggccaaatatgtggatttgagccacattgttccctctgatttcagtgccagcgctttggataacagtttctacactttcctggttcaagcattgaaagaaaaattggccccctttgtaagtgctaaagagcgtatcgttccagtctttacagggttttttggtttagttccaactggtcttctgaatggtgttggtcgtggctataccgatttatgtgccgctttgatagcagttgctgtaaatgctgatgaactacaagtttggaaggaagttgatggtatatttactgctgatcctcgtaaggttcctgaagcacgtttgctagacagtgttactccagaagaagcttctgaattaacatattatggttccgaagttatacatccttttacgatggaacaagttattagggctaagattcctattagaatcaagaatgttcaaaatccattaggtaacggtaccattatctacccagataatgtagcaaagaagggtgaatctactccaccacatcctcctgagaacttatcctcatctttctatgaaaagagaaagagaggtgccactgctatcaccaccaaaaatgacattttcgtcatcaacattcattccaataagaaaaccctatcccatggtttcctagctcaaatatttaccatcctggataagtacaagttagtcgtagatttaatatctacttctgaagttcatgtttcgatggctttgcccattccagatgcagactcattaaaatctctgagacaagctgaggaaaaattgagaattttaggttctgttgatatcacaaagaagttgtctattgtttcattagttggtaaacatatgaaacaatacatcggcattgctggtaccatgtttactactcttgctgaagaaggcatcaacattgaaatgatttctcaaggggcaaatgaaataaacatatcctgcgttatcaatgaatctgactccataaaagcgctacaatgtattcatgccaagttactaagtgagcggacaaatacttcaaaccaatttgaacatgccattgatgaacgtttagaacaattgaaaagacttggaatttag

SEQ ID NO: 26 - protein, 527 aa, Saccharomyces cerevisiae
MPMDFQPTSSHSNWVVQKFGGTSVGKFPVQIVDDIVKHYSKPDGPNNNVAVVCSARSSYTKAEGTTSRLLKCCDLASQESEFQDIIEVIRQDHIDNADRFILNPALQAKLVDDTNKELELVKKYLNASKVLGEVSSRTVDLVMSCGEKLSCLFMTALCNDRGCKAKYVDLSHIVPSDFSASALDNSFYTFLVQALKEKLAPFVSAKERIVPVFTGFFGLVPTGLLNGVGRGYTDLCAALIAVAVNADELQVWKEVDGIFTADPRKVPEARLLDSVTPEEASELTYYGSEVIHPFTMEQVIRAKIPIRIKNVQNPLGNGTIIYPDNVAKKGESTPPHPPENLSSSFYEKRKRGATAITTKNDIFVINIHSNKKTLSHGFLAQIFTILDKYKLVVDLISTSEVHVSMALPIPDADSLKSLRQAEEKLRILGSVDITKKLSIVSLVGKHMKQYIGIAGTMFTTLAEEGINIEMISQGANEINISCVINESDSIKALQCIHAKLLSERTNTSNQFEHAIDERLEQLKRLGI

SEQ ID NO: 27 - DNA, 1707 bp, Arabidopsis thaliana
  CDS (1)..(1707): Aspartate Kinase (AK1ARATH)
atggccgccaccagggtgaggtgctgccacagcaacgccgccttcaccaggctgccgctgaccaggcacaggaacagcccgaccctgccgatcagcctgaacagggtggacttcccgaccctgaagaagctgagcctgccgatcggcgacggcagcagcatcaggaaggtgagcggcagcggcagcaggaacatcgtgagggccgtgctggaggagaagaagaccgaggccatcaccgaggtggacgagaagggcatcacctgcgtgatgaagttcggcggcagcagcgtggccagcgccgagaggatgaaggaggtggccgacctgatcctgaccttcccggaggagagcccggtgatcgtgctgagcgccatgggcaagaccaccaacaacctgctgctggccggcgagaaggccgtgagctgcggcgtgagcaacgccagcgagatcgaggagctgagcatcatcaaggagctgcacatcaggaccgtgaaggagctgaacatcgacccgagcgtgatcctgacctacctggaggagctggagcagctgctgaagggcatcgccatgatgaaggagctgaccctgaggaccagggactacctggtgagcttcggcgagtgcctgagcaccaggatcttcgccgcctacctgaacaccatcggcgtgaaggccaggcagtacgacgccttcgagatcggcttcatcaccaccgacgacttcaccaacggcgacatcctggaggccacctacccggccgtggccaagaggctgtacgacgactggatgcacgacccggccgtgccgatcgtgaccggcttcctgggcaagggctggaagaccggcgccgtgaccaccctgggcaggggcggcagcgacctgaccgccaccaccatcggcaaggccctgggcctgaaggagatccaggtgtggaaggacgtggacggcgtgctgacctgcgacccgaccatctacaagagggccaccccggtgccgtacctgaccttcgacgaggccgccgagctggcctacttcggcgcccaggtgctgcacccgcagagcatgaggccggccagggagggcgagatcccggtgagggtgaagaacagctacaacccgaaggccccgggcaccatcatcaccaagaccagggacatgaccaagagcatcctgaccagcatcgtgctgaagaggaacgtgaccatgctggacatcgccagcaccaggatgctgggccaggtgggcttcctggccaaggtgttcagcatcttcgaggagctgggcatcagcgtggacgtggtggccaccagcgaggtgagcatcagcctgaccctggacccgagcaagctgtggagcagggagctgatccagcaggagctggaccacgtggtggaggagctggagaagatcgccgtggtgaacctgctgaagggcagggccatcatcagcctgatcggcaacgtgcagcacagcagcctgatcctggagagggccttccacgtgctgtacaccaagggcgtgaacgtgcagatgatcagccagggcgccagcaaggtgaacatcagcttcatcgtgaacgaggccgaggccgagggctgcgtgcaggccctgcacaagagcttcttcgagagcggcgacctgagcgagctgctgatccagccgaggctgggcaacggcagcccggtgaggaccctgcaggtggagaac

SEQ ID NO: 28 - protein, 569 aa, Arabidopsis thaliana
MAATRVRCCHSNAAFTRLPLTRHRNSPTLPISLNRVDFPTLKKLSLPIGDGSSIRKVSGSGSRNIVRAVLEEKKTEAITEVDEKGITCVMKFGGSSVASAERMKEVADLILTFPEESPVIVLSAMGKTTNNLLLAGEKAVSCGVSNASEIEELSIIKELHIRTVKELNIDPSVILTYLEELEQLLKGIAMMKELTLRTRDYLVSFGECLSTRIFAAYLNTIGVKARQYDAFEIGFITTDDFTNGDILEATYPAVAKRLYDDWMHDPAVPIVTGFLGKGWKTGAVTTLGRGGSDLTATTIGKALGLKEIQVWKDVDGVLTCDPTIYKRATPVPYLTFDEAAELAYFGAQVLHPQSMRPAREGEIPVRVKNSYNPKAPGTIITKTRDMTKSILTSIVLKRNVTMLDIASTRMLGQVGFLAKVFSIFEELGISVDVVATSEVSISLTLDPSKLWSRELIQQELDHVVEELEKIAVVNLLKGRAIISLIGNVQHSSLILERAFHVLYTKGVNVQMISQGASKVNISFIVNEAEAEGCVQALHKSFFESGDLSELLIQPRLGNGSPVRTLQVEN

SEQ ID NO: 29 - DNA, 1707 bp, Vitis v
  CDS (1)..(1707): Aspartate Kinase (A7PA24)
atggccggcgccctgcacctgtgcggcgtgaccaccccgaactgcgccgtgagcagcaggaggagcctgcactgccagccgctgtggagccagagggtggacttcaccctgagcgtggccggcggcagcaggctgtgcaggagcgtgaaggacagcagcaggaagaacgtgctgagggtgtgctgcagcggcggcagcatcgacgtgctggagaagagcaagaccgagaaccagaccaccgacggcgtgaccgagcagctgaccatcgccatgaagttcggcggcagcagcgtggccagcgccgagaggatgagggaggtggccgacctgatcctgagcttcccgaacgagaggccggtgatcgtgctgagcgccatgggcaagaccaccaacaagctgctgctggccggcgagaaggccgtgagctgcggcgtgagcaacgccagctgcatcgacgagctgaccttcatcaaggagctgcacctgaggaccgccgacgagctgggcgtggacagcagcgtgatcagcgcccacctggaggagctggagcagctgctgaagggcatcgccatgatgaaggagctgaccctgaggaccaaggactacctggtgagcttcggcgagtgcatgagcaccaggctgttcgccgcctacatgaacaagatcggcgccaaggccaggcagtacgacgccttcgacatcggcttcatcaccaccgacgacttcaccaacgccgacatcctggaggccacctacccggccgtggccaagaggctgctgggcgactggatcagcgacccggccatcccgatcgtgaccggcttcctgggcaagggctggaggagctgcgccgtgaccaccctgggcaggggcggcagcgacctgaccgccaccaccctgggcaaggccctgggcctgagggagatccaggtgtggaaggacgtggacggcgtgctgacctgcgacccgaacatctacagccacgccaagccggtggagtacctgaccttcgacgaggccgccgagctggcctacttcggcgcccaggtgctgcacccgcagagcatgaggccggccagggagggcgacatcccggtgagggtgaagaacagctacaacaggaacgccccgggcaccctgatcaccaggaccagggacatgagcaaggccgtgctgaccagcatcgtgctgaagaggaacgtgaccatgctggacatcgtgagcaccaggatgctgggccagttcggcttcctggccaaggtgttcagcatcttcgaggagctgggcatcagcgtggacgtggtggccaccagcgaggtgagcatcagcctgaccctggacccgagcaagctgtggagcagggagctgatccagcaggccagcgagctggaccacgtggtggaggagctggagaagatcgccgtggtgaacctgctgcagcacaggagcatcatcagcctgatcggcaacatccagatgagcagcctgatcctggagaaggccttccacgtgctgaggaccaagggcgtgaacgtgcagatgatcagccagggcgccagcaaggtgaacatcagcctgatcgtgaacgacgaccaggccgagaagtgcgtgagggccctgcacagcgccttcttcgagagcctgagcgaggtggacagggagtgccagagcgacaacggcagcaccagcgccttcagcaacggccac

SEQ ID NO: 30 - protein, 569 aa, Vitis v
MAGALHLCGVTTPNCAVSSRRSLHCQPLWSQRVDFTLSVAGGSRLCRSVKDSSRKNVLRVCCSGGSIDVLEKSKTENQTTDGVTEQLTIAMKFGGSSVASAERMREVADLILSFPNERPVIVLSAMGKTTNKLLLAGEKAVSCGVSNASCIDELTFIKELHLRTADELGVDSSVISAHLEELEQLLKGIAMMKELTLRTKDYLVSFGECMSTRLFAAYMNKIGAKARQYDAFDIGFITTDDFTNADILEATYPAVAKRLLGDWISDPAIPIVTGFLGKGWRSCAVTTLGRGGSDLTATTLGKALGLREIQVWKDVDGVLTCDPNIYSHAKPVEYLTFDEAAELAYFGAQVLHPQSMRPAREGDIPVRVKNSYNRNAPGTLITRTRDMSKAVLTSIVLKRNVTMLDIVSTRMLGQFGFLAKVFSIFEELGISVDVVATSEVSISLTLDPSKLWSRELIQQASELDHVVEELEKIAVVNLLQHRSIISLIGNIQMSSLILEKAFHVLRTKGVNVQMISQGASKVNISLIVNDDQAEKCVRALHSAFFESLSEVDRECQSDNGSTSAFSNGH

SEQ ID NO: 31 - DNA, 1098 bp, Sacharomyces cereviseae
  CDS (1)..(1098): Protease (ymr152w)
atgtcagacgagattgtaaccaacaaatctgttacttatgttaacaatactactccagtcacaattacatcatcggagctggacttgagatcatgttatcaagatgacgaagttgttatagaagtccatgctgctgctttgaatccaattgattttattactcaccagctttgtaactcctacatatttggcaagtatccaaagacttattctagagattacagtggtgtcatcattaaagcgggaaaggatgtcgacaatcgctggaaggttggcgacaaagtgaatggtatgtacagtcatatttatggtgaacgtggtactttaacacactacttgatactcaaccctgccaaagatgtacccatcacacatatggtggaggttccaaaggacgaaaatgacccatatgacgactttgtatatgctgctgcctggcccctaacatttggtacagccttttctacattatacgatttcaagaaagattggacttctgattcgaaagttctggttattggcgcttcaacctctgtgtcatatgcttttgtccatattgcaaaaaactacttcaacatcggcactgttgttggcatttgtagcaagaattccattgaacgcaataagaaattaggatatgactacttggttccctatgacgaaggatccatcgttgagaatgttaagaaattaaagcaaagcgtgttggaaaacgacaaatttgacatgatttttgattcagtgggtaaccatgatttcttccctgtcattgatcaatttctaaaacctaaggcgaaaaattccttttacgttacaattgcaggaaataacaaggccgattataagaatatcagttggagagatttcgtatcattgagttctattttaaaggctataaacccattcaaaaaatacaactggcgctttggacacccttatccccccaacaacttcattgaagttggcaacgaaatgatcaagaagggcacttataagccacctattgattccgtctacgagtttgatcaatataaagaagctattgacagactgatgtccaacagagctaagggtaaagtggttgttaagatgaaatga

SEQ ID NO: 32 - protein, 365 aa, Sacharomyces cereviseae
MSDEIVTNKSVTYVNNTTPVTITSSELDLRSCYQDDEVVIEVHAAALNPIDFITHQLCNSYIFGKYPKTYSRDYSGVIIKAGKDVDNRWKVGDKVNGMYSHIYGERGTLTHYLILNPAKDVPITHMVEVPKDENDPYDDFVYAAAWPLTFGTAFSTLYDFKKDWTSDSKVLVIGASTSVSYAFVHIAKNYFNIGTVVGICSKNSIERNKKLGYDYLVPYDEGSIVENVKKLKQSVLENDKFDMIFDSVGNHDFFPVIDQFLKPKAKNSFYVTIAGNNKADYKNISWRDFVSLSSILKAINPFKKYNWRFGHPYPPNNFIEVGNEMIKKGTYKPPIDSVYEFDQYKEAIDRLMSNRAKGKVVVKMK

SEQ ID NO: 33 - DNA, 1179 bp, Glycine max
  CDS (1)..(1179): Protease (GM06MC00335)
atgtctctaatagcttcactctcctccacaacttcacaactcacttcccttccttccctcccttcttccacctctccactttctcgcaaatttcccctcactttccgggaaaaaaacagaactccaatcacacctcacaagctccctttttcagcaaggcctaccaccaccagagtggtgttggtgaagtctcaggccacagcacctgcttcttctgaggctgtggcagtgacacctgtgtcttctgagatgaaggcatgggtctatggggaatatgggggtgtggatgttctgaagctggactccaatgttactgtgcctgatgtgaaggaagatcaggttctaatcaaagttgttgctgctgctcttaaccctgttgatgccaagaggaggcagggaaagtttaaggccactgattctccccttccgactgttccgggctacgatgtagcgggtgtggtggtgaaagttggtagtcaagtgaaggactttaaagtgggtgatgaagtgtatggagatgtaaatgagaaagcactagaaggacctaagcaattcgggtctttggctgagtacacagctgttgaggagaaattgttggcatcaaaaccgaagaatttggactttgctcaagctgctgctcttcctctagcaattgagactgcttatgagggtttggagaggactggattttctcctggtaaatctattcttgttctaaatggttctggaggagttggtagcctagtgattcagctagctaagcaagtttttggagcttcaagagttgctgccacttcgagtactagaaatttggacctattgaaaagcttgggagctgatttggctattgactacacaaaagagaactttgaagatttgccagaaaaatttgatgtagtttatgatgccattgggcaatgtgacagggcagtgaaggctgtgaaagaaggcggcagtgtcgtggcactcacaggtgctgttacaccacctggcttcagatttgtagttacttccaatggagctgttctgaggaaactaaacccttacttagaaagtgggaaggtgaagccaattgtagaccccaaaggtccattctcctttgataagcttgctgaagccttctcttaccttgaaacaaaccgagccactggaaaggttgttatacatccaatcccttga

SEQ ID NO: 34 - protein, 392 aa, Glycine max
MSLIASLSSTTSQLTSLPSLPSSTSPLSRKFPLTFREKNRTPITPHKLPFSARPTTTRVVLVKSQATAPASSEAVAVTPVSSEMKAWVYGEYGGVDVLKLDSNVTVPDVKEDQVLIKVVAAALNPVDAKRRQGKFKATDSPLPTVPGYDVAGVVVKVGSQVKDFKVGDEVYGDVNEKALEGPKQFGSLAEYTAVEEKLLASKPKNLDFAQAAALPLAIETAYEGLERTGFSPGKSILVLNGSGGVGSLVIQLAKQVFGASRVAATSSTRNLDLLKSLGADLAIDYTKENFEDLPEKFDVVYDAIGQCDRAVKAVKEGGSVVALTGAVTPPGFRFVVTSNGAVLRKLNPYLESGKVKPIVDPKGPFSFDKLAEAFSYLETNRATGKVVIHPIP

SEQ ID NO: 35 - DNA, 753 bp, Zea mays
  CDS (1)..(753): Protease (ZM07mc01243)
atggaccaacatgattccatggctgactcaccaaggaggcggcacaaccttttgcgtgacaaggtccaattggtgaaaaggaaagattcagatcgctatgagatcgtccgcttccatgatccactgtcttttgagaaaggcttctttgttgtgatccgtgcttgccagcttttggcacagcacaatgacgggataatatttgttggtgttgctggcccctctggtgctgggaagactgtgttcactgagaaggtggtcaatttcatgcctgatgttgctgtgatatcaatggacaactataatgacgcaactcgcatagtcgatggcaattttgatgatccacgtctgacagactatgacacactgctggaaaatatacatggtctgaaggaaggaaggtcagttcaggttccaatatatgatttcaagtcaagctgccggactggttatagaacagttgatgtccctagctcccgaattgttatcattgaaggtatttatgcactgagtgagaagttacggccagtgatagacttgcgtgtttctgtcactggtggtgttcattttgacctagtcaagagggttttaagggatatacagcgagctggccaggagcctgaagaaataattcatcagatctctgaaacggtttatccaatgtacaaggctttcatcgaaccagatttggagaccgcacatattaagatcatcaacaagttcaacccattttcagggttccaaaatcctatgtac

SEQ ID NO: 36 - protein, 251 aa, Zea mays
MDQHDSMADSPRRRHNLLRDKVQLVKRKDSDRYEIVRFHDPLSFEKGFFVVIRACQLLAQHNDGIIFVGVAGPSGAGKTVFTEKVVNFMPDVAVISMDNYNDATRIVDGNFDDPRLTDYDTLLENIHGLKEGRSVQVPIYDFKSSCRTGYRTVDVPSSRIVIIEGIYALSEKLRPVIDLRVSVTGGVHFDLVKRVLRDIQRAGQEPEEIIHQISETVYPMYKAFIEPDLETAHIKIINKFNPFSGFQNPMY

SEQ ID NO: 37 - DNA, 1095 bp, Escherichia coli
  CDS (1)..(1095): Aminomethyltransferase (b2905)
atggcacaacagactcctttgtacgaacaacacacgctttgcggcgctcgcatggtggatttccacggctggatgatgccgctgcattacggttcgcaaatcgacgaacatcatgcggtacgtaccgatgccggaatgtttgatgtgtcacatatgaccatcgtcgatcttcgcggcagccgcacccgggagtttctgcgttatctgctggcgaacgatgtggcgaagctcaccaaaagcggcaaagccctttactcggggatgttgaatgcctctggcggtgtgatagatgacctcatcgtctactactttactgaagatttcttccgcctcgttgttaactccgccacccgcgaaaaagacctctcctggattacccaacacgctgaacctttcggcatcgaaattaccgttcgtgatgacctttccatgattgccgtgcaagggccgaatgcgcaggcaaaagctgccacactgtttaatgacgcccagcgtcaggcggtggaagggatgaaaccgttctttggcgtgcaggcgggcgatctgtttattgccaccactggttataccggtgaagcgggctatgaaattgcgctgcccaatgaaaaagcggccgatttctggcgtgcgctggtggaagcgggtgttaagccatgtggcttgggcgcgcgtgacacgctgcgtctggaagcgggcatgaatctttatggtcaggagatggacgaaaccatctctcctttagccgccaacatgggctggaccatcgcctgggaaccggcagatcgtgactttatcggtcgtgaagccctggaagtgcagcgtgagcatggtacagaaaaactggttggtctggtgatgaccgaaaaaggcgtgctgcgtaatgaactgccggtacgctttaccgatgcgcagggcaaccagcatgaaggcattatcaccagcggtactttctccccgacgctgggttacagcattgcgctggcgcgcgtgccggaaggtattggcgaaacggcgattgtgcaaattcgcaaccgtgaaatgccggttaaagtgacaaaacctgtttttgtgcgtaacggcaaagccgtcgcgtga

SEQ ID NO: 38 - protein, 364 aa, Escherichia coli
MAQQTPLYEQHTLCGARMVDFHGWMMPLHYGSQIDEHHAVRTDAGMFDVSHMTIVDLRGSRTREFLRYLLANDVAKLTKSGKALYSGMLNASGGVIDDLIVYYFTEDFFRLVVNSATREKDLSWITQHAEPFGIEITVRDDLSMIAVQGPNAQAKAATLFNDAQRQAVEGMKPFFGVQAGDLFIATTGYTGEAGYEIALPNEKAADFWRALVEAGVKPCGLGARDTLRLEAGMNLYGQEMDETISPLAANMGWTIAWEPADRDFIGREALEVQREHGTEKLVGLVMTEKGVLRNELPVRFTDAQGNQHEGIITSGTFSPTLGYSIALARVPEGIGETAIVQIRNREMPVKVTKPVFVRNGKAVA

SEQ ID NO: 39 - DNA, 1227 bp, Arabidospsis thaliana
  CDS (1)..(1227): Aminomethyltransferase (AT03MContig12206)
atgagaggtgggagtctatggcagctagggcaatcaataacccgtcgtcttgctcaatctgacaagaaggttgtgtcacgtcgctactttgcctctgaagctgacctgaaaaagactgctctttacgacttccatgttgcccatggaggaaagatggttccttttgctggttggagtatgccaattcagtacaaagattcgattatggactcaacggttaactgcagggaaaatgggagtttgtttgatgttgcacatatgtgtggtttgagccttaagggtaaagattgtgttccttttcttgagacacttgtggttgctgatgtggctggtttggctcctggaactgggagcttaactgtgttcacaaacgagaaaggaggtgccattgatgactcggtgattaccaaagtgacagatgaacatatctatttggtggtcaatgctggctgtagggataaggatttggctcacattgaagaacacatgaaggctttcaaatccaaaggaggtgatgtctcgtggcatatccacgacgagagatctcttcttgcccttcagggtcctttggctgctccagtgcttcaacacctgactaaagaagacttgagcaagctttactttggcaatttccagattctggacattaatggttccacatgtttccttaccaggactgggtataccggggaagatgggtttgagatttcggttccagatgagcatgctgtggatctagcaaaagcaatcttggagaagtccgagggtaaggtaaggcttacgggtctaggagcaagagacagtctcaggttagaagcaggactttgtctatatggaaacgacatggagcaacacatttctcctgttgaagctgggctcacatgggccatagggaagcgtagaagagccgaaggtggatttcttggcgcggatgtgattctccagcagcttaaagatggacctacaatcagaagggtcggtttcttctcctcaggaccacccgcaaggtcgcatagcgaggttcatgatgagagtgggaacaagattggagagatcacaagtggagggtttagcccgaacctgaagaagaacatagccatgggatatgtgaagtcaggtcagcacaagactgggactaaagtcaagatcttggtccgtgggaaaccatatgaaggcagcatcacgaagatgccattcgtggccaccaaatactacaaaccaacatga

SEQ ID NO: 40 - protein, 408 aa, Arabidospsis thaliana
MRGGSLWQLGQSITRRLAQSDKKVVSRRYFASEADLKKTALYDFHVAHGGKMVPFAGWSMPIQYKDSIMDSTVNCRENGSLFDVAHMCGLSLKGKDCVPFLETLVVADVAGLAPGTGSLTVFTNEKGGAIDDSVITKVTDEHIYLVVNAGCRDKDLAHIEEHMKAFKSKGGDVSWHIHDERSLLALQGPLAAPVLQHLTKEDLSKLYFGNFQILDINGSTCFLTRTGYTGEDGFEISVPDEHAVDLAKAILEKSEGKVRLTGLGARDSLRLEAGLCLYGNDMEQHISPVEAGLTWAIGKRRRAEGGFLGADVILQQLKDGPTIRRVGFFSSGPPARSHSEVHDESGNKIGEITSGGFSPNLKKNIAMGYVKSGQHKTGTKVKILVRGKPYEGSITKMPFVATKYYKPT

SEQ ID NO: 41 - DNA, 696 bp, Escherichia coli
  CDS (1)..(696): Uridine Kinase (b2066)
atggcgcttcgtcgcccccttcctaacatccaggttaatcaggtcgctaaatatatgactgatcagtctcatcagtgcgtcattatcggtatcgctggcgcatcggcttccggcaagagtcttattgccagtaccctttatcgtgaattgcgtgagcaagtcggtgatgaacacatcggcgtaattcccgaagactgctattacaaagatcaaagccatctgtcgatggaagaacgcgttaagaccaactacgaccatcccagcgcgatggatcacagtctgctgcttgagcatttacaagcgttgaaacgcggctcggcaattgacctgccggtttacagctatgttgaacatacgcgtatgaaagaaacggtgacggttgagccgaagaaggtcatcattctcgaaggcattttgttgctgacggatgcgcgtttgcgtgacgaacttaacttctccattttcgttgataccccgctggatatctgcctgatgcgccgcatcaagcgtgacgttaacgagcgtgggcgttcaatggattcagtgatggcgcaatatcaaaaaaccgtgcgcccgatgttcctgcaattcattgagccttctaaacaatatgcggacattatcgtgccgcgcggcgggaaaaaccgcatcgcgatcgatatattgaaagcgaaaataagtcagttctttgaataa

SEQ ID NO: 42 - protein, 231 aa, Escherichia coli
MALRRPLPNIQVNQVAKYMTDQSHQCVIIGIAGASASGKSLIASTLYRELREQVGDEHIGVIPEDCYYKDQSHLSMEERVKTNYDHPSAMDHSLLLEHLQALKRGSAIDLPVYSYVEHTRMKETVTVEPKKVIILEGILLLTDARLRDELNFSIFVDTPLDICLMRRIKRDVNERGRSMDSVMAQYQKTVRPMFLQFIEPSKQYADIIVPRGGKNRIAIDILKAKISQFFE

SEQ ID NO: 43 - DNA, 1431 bp, Glycine max
  CDS (1)..(1431): Uridine Kinase (GM06MC13058)
atgccggaagaaacgacgtcgatcgactacgtgatggaggcggcatctgggccccacttctccggcctgagacttgacggccgtatgccgtcctccgccaccgccgccgccgcgtcctcctctgccgattctaccctaacccccgatttcctccccaatcaacccttcgtcataggtgtttcgggaggtactgcctcgggaaaaaccaccgtctgcgacatgataattcagcaacttcacgatcaccgcgttgttctcgtcaatcaggactcgttttatcgcgggttgaatcctgaagagttggaacgcgttcacgagtacaatttcgaccaccctgatgcttttgacacggagcagttgttagagtgcacgatgaagcttatcagtggccagggcgtgcatgttcccatttacgacttcaagaagcaccaacgctcttccgatagttttcgccaggtgaatgcttctgatgtgattatattggaggggattcttgtgttccacgatcagcgcgtgcgggatctgatgaacatgaagatctttgttgacacagatgctgatgtgagacttgctcgtagaattaggcgtgacacaatggagagaggcagggatataaactctgttcttgaacagtatgcaaaatttgttaagcctgcatttgatgattttgttctaccatcaaaaaagtacgctgacgtgatcattcctcgtggaggtgataatcatgttgccattgatttgattgtgcaacatatccgtacaaagctcggccaacatgatctatgcaaaatatatccaaatgcctatgttattcagtctacattccagatcaggggaatgcatacgttgattcgtgaccgagacatatcaaagcatgatttcgtattttattcagatcgacttatacgcctggttgttgagcatggtctaggtcatctgcctttcaccgaaaagcaagtggttactcccacaggatctgtttataccggtgttgatttctgcaaaaaattgtgtggtgtttcaattgttcgaagtggtgagagcatggaaaatgcacttcgtgcatgttgcaaaggtattaaaattggtaaaattttgattcaccgggatggagacaatggaaaacagcttatatatgaaaagctccccaaggatatttcagaacggcatgtcttgcttcttgaccctgtcctggccacaggtaactctgccaaccaagcaattgaattactcattcagaaaggagtaccagaaagtcacattatattcttgaatctcatttctgctcctgagggaatccactgcgtgtgtaaaaggtttccatcattgaagattgtcacgtctgagattgacattgaaataaacgaagagtatcgtgttatcccaggattgggggaatttggtgatcgatactttggtaccgatgattga

SEQ ID NO: 44 - protein, 476 aa, Glycine max
MPEETTSIDYVMEAASGPHFSGLRLDGRMPSSATAAAASSSADSTLTPDFLPNQPFVIGVSGGTASGKTTVCDMIIQQLHDHRVVLVNQDSFYRGLNPEELERVHEYNFDHPDAFDTEQLLECTMKLISGQGVHVPIYDFKKHQRSSDSFRQVNASDVIILEGILVFHDQRVRDLMNMKIFVDTDADVRLARRIRRDTMERGRDINSVLEQYAKFVKPAFDDFVLPSKKYADVIIPRGGDNHVAIDLIVQHIRTKLGQHDLCKIYPNAYVIQSTFQIRGMHTLIRDRDISKHDFVFYSDRLIRLVVEHGLGHLPFTEKQVVTPTGSVYTGVDFCKKLCGVSIVRSGESMENALRACCKGIKIGKILIHRDGDNGKQLIYEKLPKDISERHVLLLDPVLATGNSANQAIELLIQKGVPESHIIFLNLISAPEGIHCVCKRFPSLKIVTSEIDIEINEEYRVIPGLGEFGDRYFGTDD

SEQ ID NO: 45 - DNA, 1491 bp, Oriza sativa
  CDS (1)..(1491): Uridine Kinase (Os07MC01669)
atgccggagaaggcggtggacgacgtcatggactcggcggtgggggcgcacttcagcggactccgcctcgaggcgctccgcctctcctccccctccgccccgtcctccccttcctccgccaaggcggccgcggcggcggccgcccactccaacggcgccgtctacgccaacggcgtcgccgccgacgccgcggagcttgtgtcgccatccgcgctcaggcagcccttcgtcatcggggtttcgggtgggacggcgtcggggaagacgacggtgtgcgacatgatcatccagcagctgcacgaccaccgtgtcgtgctcgtcaaccaggattccttctaccgtgggttaactgctgaagaatctgcacatgcccaagattacaactttgatcaccctgatgcatttgacacagagcagcttctggaatgcatggggcagctaaaacgggcacagcctgtaaatgttcctatatatgattttaagaatcaccgacggtcctctgaaagctttagaaaggtcaatgcatcagatgtcattatattagagggcattttagtcttccatgatcaaagagttcgcaatctgatggacatgaaaatttttgtggacacagatgctgatattaggctagctcgacgaataaggcgtgatacagttgaaagaggtagagatgttagctcggtgcttgagcagtatggcaggtttgtgaagccagcatttgatgattttgttctcccttcgaaaaaatatgccgacgtgatcataccacgaggaggtgataatcatgtggcaattgacctgattgttcaacatattcgtactaagcttggtcagcatgacttgtgcaaaatctatccacatgtttatgtggttcaaacaacattccagatccgaggaatgcatactctgattcgtgacagggacatcactacacctgactttgtcttttattcagatcggttgatccgtttggtagttgagcacggactggggcatttgccatttacagaaaagcagattattactccaacagggtccatttatatgggagttgaattttgcaagaagctctgtggagtgtctattgtccgaagtggtgaaagtatggagaatgcattgcgtgcttgttgtaaaggaataaaaattggcaagattctaatacatcgtgttggagacaatggacagcaacttatataccataaactgcccatggatattgctgaacggcatgttcttcttttggatccagtgcttggtacaggtaactcagcaaatcaagctattgagcttctgataagaaaaggtgttccagaggagcgaatcatatttctcaatcttatctcggctcctgagggaattcagtgcgtctgcaagcgatttccgaggctgaagattgtaacctccgagatcgacaccgggttgagcgaggaatacagggtaatcccagggttgggagagtacggtgaccgctacttcggcacagataattga

SEQ ID NO: 46 - protein, 496 aa, Oriza sativa
MPEKAVDDVMDSAVGAHFSGLRLEALRLSSPSAPSSPSSAKAAAAAAAHSNGAVYANGVAADAAELVSPSALRQPFVIGVSGGTASGKTTVCDMIIQQLHDHRVVLVNQDSFYRGLTAEESAHAQDYNFDHPDAFDTEQLLECMGQLKRAQPVNVPIYDFKNHRRSSESFRKVNASDVIILEGILVFHDQRVRNLMDMKIFVDTDADIRLARRIRRDTVERGRDVSSVLEQYGRFVKPAFDDFVLPSKKYADVIIPRGGDNHVAIDLIVQHIRTKLGQHDLCKIYPHVYVVQTTFQIRGMHTLIRDRDITTPDFVFYSDRLIRLVVEHGLGHLPFTEKQIITPTGSIYMGVEFCKKLCGVSIVRSGESMENALRACCKGIKIGKILIHRVGDNGQQLIYHKLPMDIAERHVLLLDPVLGTGNSANQAIELLIRKGVPEERIIFLNLISAPEGIQCVCKRFPRLKIVTSEIDTGLSEEYRVIPGLGEYGDRYFGTDN

SEQ ID NO: 47 - DNA, 753 bp, Zea mays; identical to SEQ ID NO: 35
  CDS (1)..(753): Uridine Kinase (Zm07MC11423)

SEQ ID NO: 48 - protein, 251 aa, Zea mays; identical to SEQ ID NO: 36

SEQ ID NO: 49 - DNA, 684 bp, Zea mays
  CDS (1)..(684): Uridine Kinase (ZM07MC23131)
atgggttcgaaatcggtagatgaggtgctcgatgccgccgccggagtccattgctctgctctccgtctggacggtcccaacgtccagttttccgcctcctccgaggaagaaccaacgacgtctggcctggagaatggccatcaggagccctttgttattggcgttgctgggggtgcatcgtcaggcaaaagtaccgtgtgcaagatgatcattgatcagctccgtgaccagcgcgttgtggctgttacacaggagtccttttattatggattgacggatgaagaattagtccatgttcatgattataattttgatcatccagatgcatttcatacagagctacttctctcttgtatgcaaaacttgaaacatggcaaagctgtcgacatccctaattacaacttcaagacatacaagagtgtaccaaatgcaaggaaggttaatccttcagatgttattattttggaaggaattctagtttttcatgattcacgcctcagggatttgatgaacatgaagatcttcgttgatacagatgccgacgtacggttaacaaggaggatccgtcgtgataccattgacaagggtagagatatcaaagctgtgttagatcagtactcaaaatttgttaagcctgcttttgaagacttcatcctcccaacgaagaagtatgctgatattatc

SEQ ID NO: 50 - protein, 228 aa, Zea mays
MGSKSVDEVLDAAAGVHCSALRLDGPNVQFSASSEEEPTTSGLENGHQEPFVIGVAGGASSGKSTVCKMIIDQLRDQRVVAVTQESFYYGLTDEELVHVHDYNFDHPDAFHTELLLSCMQNLKHGKAVDIPNYNFKTYKSVPNARKVNPSDVIILEGILVFHDSRLRDLMNMKIFVDTDADVRLTRRIRRDTIDKGRDIKAVLDQYSKFVKPAFEDFILPTKKYADII

SEQ ID NO: 51 - DNA, 1662 bp, Synechocystis sp. PCC 6803
  CDS (1)..(1662): L-Aspartate Oxidase
atgggggcaaatcagttcgatgtggtggtggtgggctccggagcggcaggcctttatgcctgtctttgtttgcctggtcattaccgtgtggctctggttaccaaggcggaactgaaaacgggggccagtgattgggcccagggagggattgcggccgctattgccccgacggattctccccaatctcactacgaagataccttagctgctggagcgggtttatgtgatggggaagcggtggatttcctagtgaatcatgccccccaggcgatcgccgagttggtgcagttcggtgttagttttgatcgccatgggcagcatctggctttaacgttggaggcggcccattcccaaccgagggtactccatgcggcggacactacggggcgggccattgtttcgacgttgatgggacaggtgcaagctcggcccaatgtggaaattttttcccaggcgatcgccctgtctttaaatattgagccaacaacgggccattgtcggggcattcaggtttttgtcaatcaaaccattgaaactttccactcccgagcggtgttattggccaccgggggcggcggtcaagtgtttgcccaaactaccaatcccaaagttagtactggtgacggcattgccctcgcttggcgatcgggggcccaggtgcgggatttagaattctttcaatttcatcccaccgctttaactaaaccgggtgtgccccattttttgatcagtgaagcagtgcggggggaaggggcccatctgctcgatcgccaggggaaaagatttgcttttgattaccatcccaggggagaactagctcccagggacgtggttagtcgggcaattttccaacatttagccaatacagaaaaagatcccacccaagccactgtttttttggatctgagtcccattgagccggagcgcatccaaaggcgttttcccaatattattcgccgttgtttgcactggggggtggacatttttcgtgaaccaattcccgttgccccagcggcccattattggatggggggcattaccacagatattaattgtcaaaccactattccaggcttgtatgcgttgggggaaaccgccagcactggggtccatggagccaatcgcctggccagtaattctctgctggaatgcattgtttttgctagtcaattaagaaatttgagcttaccgccccttgcctccagtgactccaacgttaatcaatcgataaaggaaattcgactagacaccaccaatgacctagccctcctaaaccattggcgcagtgaattacctcgcttgatgtggcaaaccgccggcatttgtcgccaagctgaaactctccaaatggcgatcgccaaattggaacaatggcaagaacaatggcagcagctcagcagcagtaagcttttagctcacctaccagaggatcaaaaaattagcctaagtggcccagggctgaatgaatttatgcagttgtgggccgaaacccataatctgctggatatagcacaattaattttgactagtgccctctttcgggaggaaagccgggggggacattatcggttagattgtcctgataccaaaagagaatggcaaagccacactgtcattgaaggaacgagggtttttctccaacccagccagagtcaagactaa

SEQ ID NO: 52 - protein, 553 aa, Synechocystis sp. PCC 6803
MGANQFDVVVVGSGAAGLYACLCLPGHYRVALVTKAELKTGASDWAQGGIAAAIAPTDSPQSHYEDTLAAGAGLCDGEAVDFLVNHAPQAIAELVQFGVSFDRHGQHLALTLEAAHSQPRVLHAADTTGRAIVSTLMGQVQARPNVEIFSQAIALSLNIEPTTGHCRGIQVFVNQTIETFHSRAVLLATGGGGQVFAQTTNPKVSTGDGIALAWRSGAQVRDLEFFQFHPTALTKPGVPHFLISEAVRGEGAHLLDRQGKRFAFDYHPRGELAPRDVVSRAIFQHLANTEKDPTQATVFLDLSPIEPERIQRRFPNIIRRCLHWGVDIFREPIPVAPAAHYWMGGITTDINCQTTIPGLYALGETASTGVHGANRLASNSLLECIVFASQLRNLSLPPLASSDSNVNQSIKEIRLDTTNDLALLNHWRSELPRLMWQTAGICRQAETLQMAIAKLEQWQEQWQQLSSSKLLAHLPEDQKISLSGPGLNEFMQLWAETHNLLDIAQLILTSALFREESRGGHYRLDCPDTKREWQSHTVIEGTRVFLQPSQSQD

SEQ ID NO: 53 - DNA, 986 bp, Petroselinum crispum
  promoter (1)..(986)
ctagaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcag

SEQ ID NO: 54 - DNA, 1112 bp, Agrobacterium tumefaciens
  promoter (1)..(1112)
ggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccgcgagctggtcaatcccattgcttttgaagcagctcaacattgatctctttctcgatcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcga

SEQ ID NO: 55 - DNA, 695 bp, Vicia fava
  promoter (1)..(695)
ctagactgcagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcataccattaattaag

SEQ ID NO: 56 - DNA, 674 bp, Vicia fava
  promoter (1)..(674): variant
caaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagc

SEQ ID NO: 57 - DNA, 102 bp, Arabidopsis thaliana
  CDS (1)..(102)
  transit_peptide (1)..(102)
atgcagaggtttttctccgccagatcgattctcggttacgccgtcaagacgcggaggaggtctttctcttctcgttcttcgtctctcctttgctcttccatg

SEQ ID NO: 58 - protein, 34 aa, Arabidopsis thaliana
MQRFFSARSILGYAVKTRRRSFSSRSSSLLCSSM

SEQ ID NO: 59 - DNA, 102 bp, Arabidopsis thaliana
  CDS (1)..(102)
  transit_peptide (1)..(102)
atgcagaggtttttctccgccagatcgattctcggttacgccgtcaagacgcggaggaggtctttctcttctcgttcttcggaattccagctgaccaccatg

SEQ ID NO: 60 - protein, 34 aa, Arabidopsis thaliana
MQRFFSARSILGYAVKTRRRSFSSRSSEFQLTTM